SEQUENCE LISTING (8 sequences)

SEQ ID NO: 1 - DNA, 20 bp, SARS-CoV-2
gaccccaaaatcagcgaaat

SEQ ID NO: 2 - DNA, 24 bp, SARS-CoV-2
tctggttactgccagttgaatctg

SEQ ID NO: 3 - DNA, 20 bp, SARS-CoV-2
gtccttccctcagtcagcac

SEQ ID NO: 4 - DNA, 20 bp, SARS-CoV-2
atggcaggagcagttgtgaa

SEQ ID NO: 5 - DNA, 20 bp, Homo sapiens
gtaacccgttgaaccccatt

SEQ ID NO: 6 - DNA, 20 bp, Homo sapiens
ccatccaatcggtagtagcg

SEQ ID NO: 7 - DNA, 20 bp, Mus musculus
gcaattattccccatgaacg

SEQ ID NO: 8 - DNA, 20 bp, Mus musculus
ggcctcactaaaccatccaa

(19) United States
(12) Patent Application Publication
(10) Pub. No.: US 2023/0285435 A1
(43) Pub. Date: Sep. 14, 2023

(54) COMPOSITIONS AND METHODS FOR TREATING VIRAL INFECTIONS

(71) Applicant: The Regents of the University of California, Oakland, CA (US)

(72) Inventor: Julia Schaletzky, Berkeley, CA (US)

(21) Appl. No.: 18/014,705

(22) PCT Filed: Jul. 15, 2021
(86) PCT No.: PCT/US21/41827

§ 371 Date: Jan. 5, 2023

Related U.S. Application Data
(60) Provisional application No. 63/053,208, filed on Jul. 17, 2020.
(60) Provisional application No. 63/130,117, filed on Dec. 23, 2020.

(51) Int. Cl. A61K 31/7072; A61K 31/4184; A61K 31/4985; A61K 31/4965; A61K 31/506; A61K 31/4025; A61K 31/4439; A61P 31/14; A61K 9/00

(52) U.S. Cl. CPC A61K 31/7072; A61K 31/4184; A61K 31/4985; A61K 31/4965; A61K 31/506; A61K 31/4025; A61K 31/4439; A61P 31/14; A61K 9/0019

(57) ABSTRACT

The present disclosure provides methods of treating an RNA virus infection. The methods comprise administering combined effective amounts of an RNA-dependent RNA polymerase inhibitor, such as remdesivir, and a second therapeutic agent for treating infection with an RNA virus.

### CROSS-REFERENCE

This application claims the benefit of U.S. Provisional Patent Application No. 63/053,208, filed Jul. 17, 2020, and U.S. Provisional Patent Application No. 63/130,117, filed Dec. 23, 2020, which applications are incorporated herein by reference in their entirety.

### INTRODUCTION

As of June 2020, the pandemic caused by SARS-CoV-2 infections (Coronaviral Disease 2019 (Covid-19)) caused about 9 million infections and about 460,000 deaths worldwide. The pandemic is expected to expand in the late 2020, particularly, because of the lack of a therapeutically effective treatment for the disease.

Remdesivir has received Emergency Authorization by the U.S. Food and Drug Administration (FDA) to be used as a therapeutic for Covid-19. A nucleoside analog drug first developed to treat disease caused by Ebola virus infection, remdesivir is an antiviral effective against SARS-CoV1, Middle East Respiratory Syndrome Coronavirus (MERS-CoV), and SARS-CoV2 in cell assays, albeit with moderate potency.

There is a need for treatments for Covid-19, the disease caused by SARS-CoV2.

### SUMMARY

The present disclosure provides methods of treating an RNA virus infection. The methods comprise administering combined effective amounts of an RNA-dependent RNA polymerase inhibitor, such as remdesivir, and a second therapeutic agent for treating infection with an RNA virus.

### BRIEF DESCRIPTION OF THE DRAWINGS

FIG. 1 provides synergy scores for compounds tested in combination with remdesivir.

FIG. 2 shows that certain compounds or combinations or compounds exhibit synergistic effect with remdesivir. Compared to when administered alone, these compounds or combinations of compounds, when administered in combination with remdesivir exhibit lower EC50 and higher inhibition of virus-induced cytopathic effects.

FIG. 3A-3H depict the effect of combinations of agents on four different strains of SARS-CoV-2.

### DEFINITIONS

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may be predisposed to acquiring the disease or symptom but has not yet been diagnosed as having it; (b) inhibiting the disease or symptom, i.e., arresting its development; and/or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. A subject therapy will in some cases be administered before the symptomatic stage of the disease (e.g., after diagnosis of a viral infection, but before symptoms appear). A subject therapy will in some cases be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, etc.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “an RNA-dependent RNA polymerase inhibitor” includes a plurality of such RNA-dependent RNA polymerase inhibitors and reference to “the RNA virus” includes reference to one or more RNA viruses and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides methods of treating an RNA virus infection. The methods comprise administering combined effective amounts of an RNA-dependent RNA polymerase inhibitor, such as remdesivir, and a second therapeutic agent (i.e., at least one additional therapeutic agent) for treating infection with an RNA virus.

The present disclosure provides a method of treating a viral infection caused by an RNA virus, the method comprising administering a first therapeutic agent and a second therapeutic agent in combined effective amounts. The first therapeutic agent is an inhibitor of an RNA-dependent RNA polymerase (RdRp), such as remdesivir. The second therapeutic agent (the at least one additional therapeutic agent) is an agent other than an RdRp inhibitor. In some cases, three additional therapeutic agents are administered.

The first therapeutic agent and the second therapeutic agent (the at least one additional therapeutic agent) are in some cases administered in synergistically effective amounts. Thus, e.g., in some cases, an effective amount of the RdRp inhibitor that is administered in a combination therapy of the present disclosure is lower than the amount of the RdRp inhibitor that is effective when administered as monotherapy. For example, in some cases, the amount of the RdRp inhibitor that, when administered in a combination therapy of the present disclosure, is effective to treat an RNA virus infection, is at least 10%, at least 15%, at least 20%, at least 25% at least 30%, at least 35%, at least 40%, at least 45%, at least 50%, or more than 50%, lower than the amount that, when administered in monotherapy, is effective to treat the RNA virus infection.

For example, in some cases, an effective amount of remdesivir that is administered in a combination therapy of the present disclosure is lower than the amount of remdesivir that is effective when administered as monotherapy. For example, in some cases, the amount of remdesivir that, when administered in a combination therapy of the present disclosure, is effective to treat Covid-19, is at least 10%, at least 15%, at least 20%, at least 25% at least 30%, at least 35%, at least 40%, at least 45%, at least 50%, at least 60%, at least 70%, at least 80%, at least 90%, or more than 90%, lower than the amount that, when administered in monotherapy, is effective to treat Covid-19.

As an example, in some cases, an amount of remdesivir that is administered in monotherapy is as follows: where the individual weighs 40 kg or more, remdesivir is administered in a single loading dose of 200 mg on Day 1 followed by a once-daily maintenance dose of 100 mg from Day 2. In such cases, according to a combination therapy method of the present disclosure, an effective amount of remdesivir, for an individual weighing 40 kg or more, can be from about 50 mg to about 190 mg; e.g., a loading dose (Day 1) of from about 80 mg to about 90 mg, from about 90 mg to about 100 mg, from about 100 mg to about 110 mg, from about 110 mg to about 120 mg, from about 120 mg to about 130 mg, from about 130 mg to about 140 mg, from about 140 mg to about 150 mg, from about 150 mg to about 160 mg, from about 160 mg to about 170 mg, from about 170 mg to about 180 mg, or from about 180 mg to about 190 mg; and a daily dose (starting on Day 2) of from about 50 mg to about 60 mg, from about 60 mg to about 70 mg, from about 70 mg to about 80 mg, or from about 80 mg to about 90 mg per day. In some cases, an effective amount of remdesivir, for an individual weighing 40 kg or more, is an amount that, when administered in a combination therapy of the present disclosure, is at least 10%, at least 15%, at least 20%, at least 25% at least 30%, at least 35%, at least 40%, at least 45%, at least 50%, at least 60%, at least 70%, at least 80%, at least 90%, or more than 90%, lower than the amount used in monotherapy; for example, the loading dose is at least 10%, at least 15%, at least 20%, at least 25% at least 30%, at least 35%, at least 40%, at least 45%, at least 50%, at least 60%, at least 70%, at least 80%, at least 90%, or more than 90%, lower than a loading dose of 200 mg; and the maintenance dose is at least 10%, at least 15%, at least 20%, at least 25% at least 30%, at least 35%, at least 40%, at least 45%, at least 50%, at least 60%, at least 70%, at least 80%, at least 90%, or more than 90%, lower than a maintenance dose of 100 mg.

As another example, in some cases, an amount of remdesivir that is administered in monotherapy is as follows: the individual weighs from 3.5 kg to 40 kg, remdesivir is administered in a single loading dose of 5 mg/kg on Day 1, followed by a once daily dose of 2.5 mg/kg from Day 2. In such cases, according to a combination therapy method of the present disclosure, an effective amount of remdesivir, for an individual weighing less than 40 kg (e.g., from 3.5 kg to 39 kg), can be from about 2.25 mg/kg to about 4.5 mg/kg; e.g., a loading dose (Day 1) of from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg; and a daily dose (starting on Day 2) of from about 0.25 mg/kg to about 0.50 mg/kg, from about 0.50 mg/kg to about 0.75 mg/kg, from about 0.75 mg/kg to about 1.0 mg/kg, from about 1.0 mg/kg to about 1.25 mg/kg, from about 1.25 mg/kg to about 1.5 mg/kg, from about 1.5 mg/kg to about 1.75 mg/kg, from about 1.75 mg/kg to about 2.0 mg/kg, or from about 2.0 mg/kg to about 2.25 mg/kg.

In some cases, an effective amount of remdesivir, for an individual weighing for an individual weighing less than 40 kg (e.g., from 3.5 kg to 39 kg), is an amount that, when administered in a combination therapy of the present disclosure, is at least 10%, at least 15%, at least 20%, at least 25% at least 30%, at least 35%, at least 40%, at least 45%, at least 50%, at least 60%, at least 70%, at least 80%, at least 90%, or more than 90%, lower than the amount used in monotherapy; for example, the loading dose is at least 10%, at least 15%, at least 20%, at least 25% at least 30%, at least 35%, at least 40%, at least 45%, at least 50%, at least 60%, at least 70%, at least 80%, at least 90%, or more than 90%, lower than a loading dose of 5 mg/kg; and the maintenance dose is at least 10%, at least 15%, at least 20%, at least 25% at least 30%, at least 35%, at least 40%, at least 45%, at least 50%, at least 60%, at least 70%, at least 80%, at least 90%, or more than 90%, lower than a maintenance dose of 2.5 mg/kg.

In some cases, “combined effective amounts” of a first therapeutic agent and a second therapeutic agent, are amounts that, when administered to an individual (an individual having an RNA virus infection; e.g., having a disease caused by an RNA virus) according to a method of the present disclosure, are effective to reduce at least one symptom of an RNA virus infection by at least 10% at least 15%, at least 20%, at least 25%, at least 30%, at least 35%, at least 40%, at least 45%, at least 50%, at least 55%, at least 60%, at least 70%, at least 80%, or at least 90%, compared to the severity of the symptom before administration of the first therapeutic agent and a second therapeutic agent. Symptoms include fever, cough, shortness of breath, and the like.

In some cases, “combined effective amounts” of a first therapeutic agent and a second therapeutic agent, are amounts that, when administered to an individual (an individual having an RNA virus infection; e.g., having a disease caused by an RNA virus) according to a method of the present disclosure, are effective to reduce viral load (the viral load of the disease-causing RNA virus) by at least 10% at least 15%, at least 20%, at least 25%, at least 30%, at least 35%, at least 40%, at least 45%, at least 50%, at least 55%, at least 60%, at least 70%, at least 80%, or at least 90%, compared to the viral load in the individual before administration of the first therapeutic agent and a second therapeutic agent.

In some cases, “combined effective amounts” of a first therapeutic agent and a second therapeutic agent, are amounts that, when administered to an individual (an individual having an RNA virus infection; e.g., having a disease caused by an RNA virus) according to a method of the present disclosure, are effective to reduce the length of time the individual is on a ventilator by at least 10% at least 15%, at least 20%, at least 25%, at least 30%, at least 35%, at least 40%, at least 45%, at least 50%, at least 55%, at least 60%, at least 70%, at least 80%, or at least 90%, compared to the length of time the individual is on the ventilator in the absence of treatment with the first therapeutic agent and a second therapeutic agent.

### First Therapeutic Agent

As noted above, the first therapeutic agent is an RdRp inhibitor. Suitable RdRp inhibitors include, e.g., sofosbuvir, remdesivir, ribavirin, favipiravir, pimodivir, or baloxavir. In some cases, the RdRp inhibitor is remdesivir.

Remdesivir has the following structure:

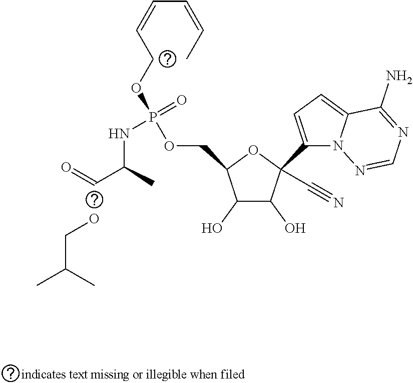

### Second Therapeutic Agent

In some cases, the second therapeutic agent is selected from: a Hepatitis C Virus (HCV) NS5A inhibitor, an HCV therapeutic agent (e.g., an HCV inhibitor), an inhibitor of B-Raf, a proton pump inhibitor (PPI), an angiotensin II receptor blocker or antagonist, a prostacyclin receptor agonist, a calcium channel blocker, a dihydropyridine-type calcium channel blocker, a leukotriene receptor antagonist, a retinoid that selectively activates a retinoid X receptor, a corticosteroid, a selective inhibitor of phosphodiesterase type 3, an arginine vasopressin (AVP) receptor antagonist, an agonist of the progesterone receptor (PR), a selective inhibitor of cyclooxygenase-2, an inhibitor of the sodium glucose co-transporter-2 (SGLT-2), a Niemann-Pick C1-like 1 (NPC1L1) protein blocker, an inhibitor of isocitrate dehydrogenase-1 (IDH1), a protein folding chaperone, a corticosteroid, an antagonist of P2Y₁₂ adenosine diphosphate (ADP) receptor, and an angiotensin converting enzyme (ACE) inhibitor.

### HCV Inhibitors

In some cases, the second therapeutic agent is an HCV therapeutic agent, i.e., a therapeutic agent that treats an HCV infection. In some cases, the second therapeutic agent is Velpatasvir. In some cases, the second therapeutic agent is Elbasvir.

Velpatasvir has the following structure:

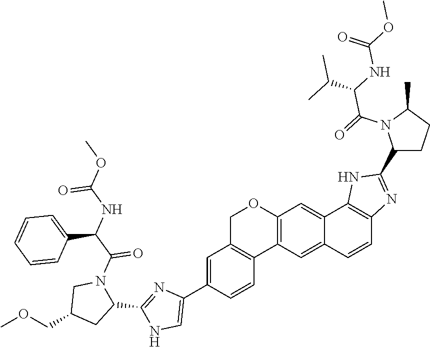

Elbasvir has the following structure:

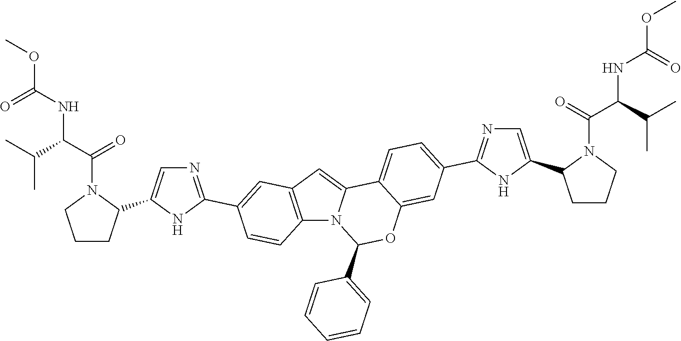

In some cases, a method of the present disclosure comprises administering: a) remdesivir; and b) a therapeutic agent for treating an HCV infection, in combined effective amounts to an individual having an RNA virus infection, e.g., an individual having a disease caused by the RNA virus infection. In some cases, a method of the present disclosure comprises administering: a) remdesivir; and b) Velpatasvir in combined effective amounts to an individual having an RNA virus infection, e.g., an individual having a disease caused by the RNA virus infection. In some cases, the individual is infected with SARS-CoV2. In some cases, the individual has Covid-19. In some cases, the method further comprises administering to the individual an amount of sofosbuvir. Sofosbuvir has the following structure:

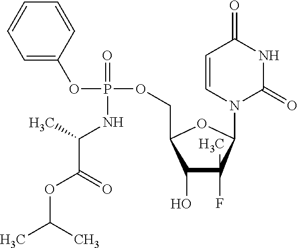

In some cases, a method of the present disclosure comprises administering: a) remdesivir; and b) Elbasvir in combined effective amounts to an individual having an RNA virus infection, e.g., an individual having a disease caused by the RNA virus infection. In some cases, the individual is infected with SARS-CoV2. In some cases, the individual has Covid-19. In some cases, the method further comprises administering to the individual an amount of sofosbuvir. In some cases, the method further comprises administering to the individual an amount of Grazoprevir, boceprevir, simeprevir, or elaprevir. In some cases, the method further comprises administering to the individual an amount of Grazoprevir. Grazoprevir has the following structure:

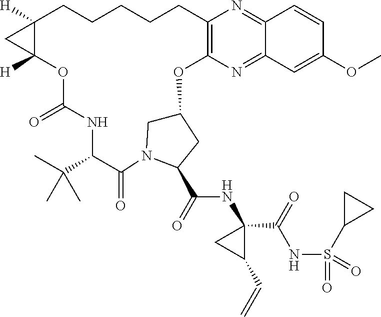 [SMILES: C=C[C@@H]1C[C@]1(NC(=O)[C@@H]1C[C@@H]2CN1C(=O)[C@H](C(C)(C)C)NC(=O)O[C@@H]1C[C@H]1CCCCCc1nc3ccc(OC)cc3nc1O2)C(=O)NS(=O)(=O)C1CC1]

### B-Raf Inhibitors

In some cases, the second therapeutic agent is an inhibitor of the enzyme B-Raf. In some cases, the second therapeutic agent is Vemurafenib. In some cases, the second therapeutic agent is encorafenib. In some cases, the second therapeutic agent is Dabrafenib. In some cases, a method of the present disclosure comprises administering: a) remdesivir; and b) a B-Raf inhibitor in combined effective amounts to an individual having an RNA virus infection, e.g., an individual having a disease caused by the RNA virus infection. In some cases, a method of the present disclosure comprises administering: a) remdesivir; and b) Dabrafenib in combined effective amounts to an individual having an RNA virus infection, e.g., an individual having a disease caused by the RNA virus infection. In some cases, the individual is infected with SARS-CoV2. In some cases, the individual has Covid-19. Dabrafenib has the following structure:

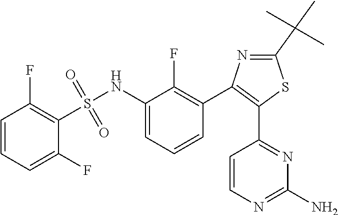 [SMILES: CC(C)(C)c1nc(-c2cccc(NS(=O)(=O)c3c(F)cccc3F)c2F)c(-c2ccnc(N)n2)s1]

Vemurafenib has the following structure:

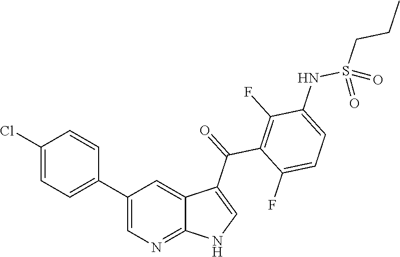 [SMILES: CCCS(=O)(=O)Nc1ccc(F)c(C(=O)c2c[nH]c3ncc(-c4ccc(Cl)cc4)cc23)c1F]

### Proton-Pump Inhibitors

In some cases, the second therapeutic agent is a proton pump inhibitor (PPI). PPIs are known in the art and include, e.g., Omeprazole, lansoprazole, pantoprazole, rabeprazole, exomeprazole, and dexlansoprazole. In some cases, the PPI is Omeprazole. In some cases, a method of the present disclosure comprises administering: a) remdesivir; and b) a PPI in combined effective amounts to an individual having an RNA virus infection, e.g., an individual having a disease caused by the RNA virus infection. In some cases, a method of the present disclosure comprises administering: a) remdesivir; and b) Omeprazole in combined effective amounts to an individual having an RNA virus infection, e.g., an individual having a disease caused by the RNA virus infection. In some cases, the individual is infected with SARS-CoV2. In some cases, the individual has Covid-19. Omeprazole has the following structure:

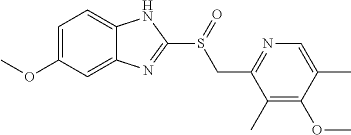

### Angiotensin II Receptor Blockers and Antagonists

In some cases, the second therapeutic agent is an angiotensin II receptor blocker. In some cases, the second therapeutic agent is an angiotensin II receptor antagonist. Angiotensin II receptor blockers include Azilsartan, Candesartan, Eprosartan, Irbesartan, Losartan, Olmesartan, Telmisartan, and Valsartan.

In some cases, a method of the present disclosure comprises administering: a) remdesivir; and b) an angiotensin II receptor blocker in combined effective amounts to an individual having an RNA virus infection, e.g., an individual having a disease caused by the RNA virus infection. In some cases, the angiotensin II receptor blocker is Telmisartan. In some cases, a method of the present disclosure comprises administering: a) remdesivir; and b) Telmisartan in combined effective amounts to an individual having an RNA virus infection, e.g., an individual having a disease caused by the RNA virus infection. In some cases, the individual is infected with SARS-CoV2. In some cases, the individual has Covid-19. Telmisartan has the following structure:

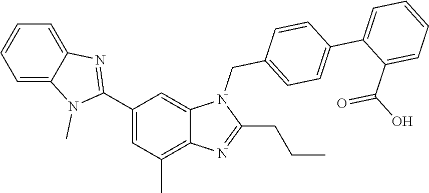 [SMILES: CCCc1nc2c(C)cc(-c3nc4ccccc4n3C)cc2n1Cc1ccc(-c2ccccc2C(=O)O)cc1]

In some cases, a method of the present disclosure comprises administering: a) remdesivir; and b) an angiotensin II receptor antagonist in combined effective amounts to an individual having an RNA virus infection, e.g., an individual having a disease caused by the RNA virus infection. In some cases, the angiotensin II receptor antagonist is Irbesartan. In some cases, a method of the present disclosure comprises administering: a) remdesivir; and b) Irbesartan in combined effective amounts to an individual having an RNA virus infection, e.g., an individual having a disease caused by the RNA virus infection. In some cases, the individual is infected with SARS-CoV2. In some cases, the individual has Covid-19. Irbesartan has the following structure:

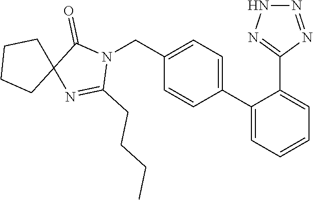 [SMILES: CCCCC1=NC2(CCCC2)C(=O)N1Cc1ccc(-c2ccccc2-c2nn[nH]n2)cc1]

### Prostacyclin Receptor Agonists

In some cases, the second therapeutic agent is a prostacyclin receptor agonist. In some cases, the prostacyclin receptor agonist is Selexipag. In some cases, a method of the present disclosure comprises administering: a) remdesivir; and b) a prostacyclin receptor agonist in combined effective amounts to an individual having an RNA virus infection, e.g., an individual having a disease caused by the RNA virus infection. In some cases, a method of the present disclosure comprises administering: a) remdesivir; and b) Selexipag in combined effective amounts to an individual having an RNA virus infection, e.g., an individual having a disease caused by the RNA virus infection. In some cases, the individual is infected with SARS-CoV2. In some cases, the individual has Covid-19. Selexipag has the following structure:

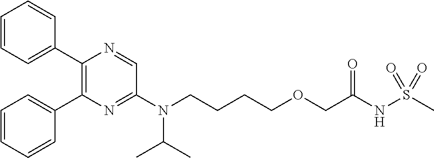 [SMILES: CC(C)N(CCCCOCC(=O)NS(C)(=O)=O)c1cnc(-c2ccccc2)c(-c2ccccc2)n1]

### Calcium Channel Blockers

In some cases, the second therapeutic agent is a calcium channel blocker. Calcium channel blockers include, e.g., Amlodipine, Diltiazem, Felodipine, Isradipine, Nicardipine, Nifedipine, Nisoldipine, and Verapamil. In some cases, the second therapeutic agent is a dihydropyridine-type calcium channel blocker.

In some cases, a method of the present disclosure comprises administering: a) remdesivir; and b) a calcium channel blocker in combined effective amounts to an individual having an RNA virus infection, e.g., an individual having a disease caused by the RNA virus infection. In some cases, a method of the present disclosure comprises administering: a) remdesivir; and b) Nifedipine in combined effective amounts to an individual having an RNA virus infection, e.g., an individual having a disease caused by the RNA virus infection. In some cases, the individual is infected with SARS-CoV2. In some cases, the individual has Covid-19. Nifedipine has the following structure:

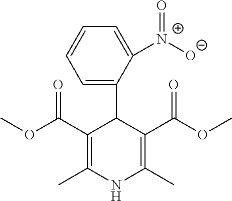

In some cases, a method of the present disclosure comprises administering: a) remdesivir; and b) Nimodipine in combined effective amounts to an individual having an RNA virus infection, e.g., an individual having a disease caused by the RNA virus infection. In some cases, the individual is infected with SARS-CoV2. In some cases, the individual has Covid-19. Nimodipine has the following structure:

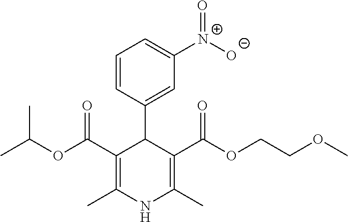 [SMILES: COCCOC(=O)C1=C(C)NC(C)=C(C(=O)OC(C)C)C1c1cccc([N+](=O)[O-])c1]

### Leukotriene Receptor Antagonists

In some cases, the second therapeutic agent is a leukotriene receptor antagonist. In some cases, the leukotriene receptor antagonist is Montelukast. In some cases, the leukotriene receptor antagonist is Zafirlukast.

In some cases, a method of the present disclosure comprises administering: a) remdesivir; and b) a leukotriene receptor antagonist in combined effective amounts to an individual having an RNA virus infection, e.g., an individual having a disease caused by the RNA virus infection. In some cases, a method of the present disclosure comprises administering: a) remdesivir; and b) Zafirlukast in combined effective amounts to an individual having an RNA virus infection, e.g., an individual having a disease caused by the RNA virus infection. In some cases, the individual is infected with SARS-CoV2. In some cases, the individual has Covid-19. Zafirlukast has the following structure:

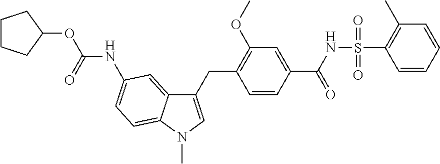 [SMILES: COc1cc(C(=O)NS(=O)(=O)c2ccccc2C)ccc1Cc1cn(C)c2ccc(NC(=O)OC3CCCC3)cc12]

### Retinoid X Receptor Activators

In some cases, the second therapeutic agent is a retinoid X receptor (RXR) activator. In some cases, the second therapeutic agent is a retinoid that selectively activates retinoid X receptors (RXRs) (as opposed to the retinoic acid receptors). In some cases, the RXR activator is Bexarotene. In some cases, a method of the present disclosure comprises administering: a) remdesivir; and b) an RXR activator in combined effective amounts to an individual having an RNA virus infection, e.g., an individual having a disease caused by the RNA virus infection. In some cases, a method of the present disclosure comprises administering: a) remdesivir; and b) Bexarotene in combined effective amounts to an individual having an RNA virus infection, e.g., an individual having a disease caused by the RNA virus infection. In some cases, the individual is infected with SARS-CoV2. In some cases, the individual has Covid-19. Bexarotene has the following structure:

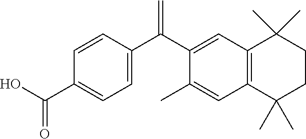 [SMILES: C=C(c1ccc(C(=O)O)cc1)c1cc2c(cc1C)C(C)(C)CCC2(C)C]

### Corticosteroids

In some cases, the second therapeutic agent is a corticosteroid. In some cases, the corticosteroid is a glucocorticoid. Corticosteroids include, e.g., bethamethasone, budesonide, prednisone, prednisolone, triamcinolone, methylprednisolone, dexamethasone, clobetasol propionate, and meprednisone.

In some cases, a method of the present disclosure comprises administering: a) remdesivir; and b) a corticosteroid in combined effective amounts to an individual having an RNA virus infection, e.g., an individual having a disease caused by the RNA virus infection.

In some cases, a method of the present disclosure comprises administering: a) remdesivir; and b) Budesonide in combined effective amounts to an individual having an RNA virus infection, e.g., an individual having a disease caused by the RNA virus infection. In some cases, the individual is infected with SARS-CoV2. In some cases, the individual has Covid-19. Budesonide has the following structure:

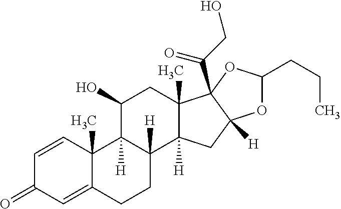

In some cases, a method of the present disclosure comprises administering: a) remdesivir; and b) Clobetasol propionate in combined effective amounts to an individual having an RNA virus infection, e.g., an individual having a disease caused by the RNA virus infection. In some cases, the individual is infected with SARS-CoV2. In some cases, the individual has Covid-19. Clobetasol propionate has the following structure:

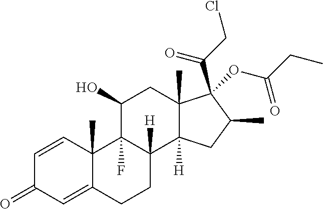

In some cases, a method of the present disclosure comprises administering: a) remdesivir; and b) meprednisone in combined effective amounts to an individual having an RNA virus infection, e.g., an individual having a disease caused by the RNA virus infection. In some cases, the individual is infected with SARS-CoV2. In some cases, the individual has Covid-19. Meprednisone has the following structure:

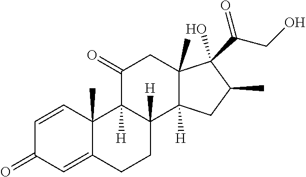

In some cases, a method of the present disclosure comprises administering: a) remdesivir; and b) methylprednisolone in combined effective amounts to an individual having an RNA virus infection, e.g., an individual having a disease caused by the RNA virus infection. In some cases, the individual is infected with SARS-CoV2. In some cases, the individual has Covid-19. Methylprednisolone has the following structure:

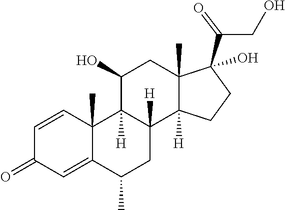

### Phosphodiesterase Type 3 (PDE₃) Inhibitors

In some cases, the second therapeutic agent is a PDE₃ inhibitor. In some cases, the second therapeutic agent is a selective PDE₃ inhibitor. PDE₃ inhibitors include, e.g., amrinone, cilostazol, milrinone, and enoximone.

In some cases, a method of the present disclosure comprises administering: a) remdesivir; and b) a PDE₃ inhibitor in combined effective amounts to an individual having an RNA virus infection, e.g., an individual having a disease caused by the RNA virus infection. In some cases, a method of the present disclosure comprises administering: a) remdesivir; and b) cilostazol in combined effective amounts to an individual having an RNA virus infection, e.g., an individual having a disease caused by the RNA virus infection. In some cases, the individual is infected with SARS-CoV2. In some cases, the individual has Covid-19. Cilostazol has the following structure:

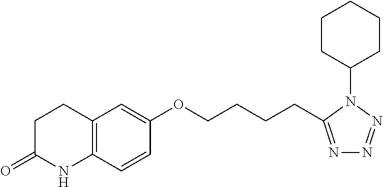 [SMILES: O=C1CCc2cc(OCCCCc3nnnn3C3CCCCC3)ccc2N1]

### Arginine Vasopressin Receptor Antagonists

In some cases, the second therapeutic agent is an arginine vasopressin receptor antagonist. In some cases, the second therapeutic agent is an arginine vasopressin receptor antagonist with affinity for human VIA and V2 receptors. Arginine vasopressin receptor antagonists include, e.g., conivaptan (e.g., conivaptan hydrochloride), tolvaptan, lixivaptan, and satavaptan.

In some cases, a method of the present disclosure comprises administering: a) remdesivir; and b) an arginine vasopressin receptor antagonist in combined effective amounts to an individual having an RNA virus infection, e.g., an individual having a disease caused by the RNA virus infection. In some cases, a method of the present disclosure comprises administering: a) remdesivir; and b) conivaptan hydrochloride in combined effective amounts to an individual having an RNA virus infection, e.g., an individual having a disease caused by the RNA virus infection. In some cases, the individual is infected with SARS-CoV2. In some cases, the individual has Covid-19. Conivaptan hydrochloride has the following structure:

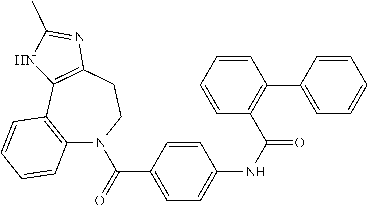 [SMILES: Cc1nc2c([nH]1)-c1ccccc1N(C(=O)c1ccc(NC(=O)c3ccccc3-c3ccccc3)cc1)CC2]

### Progesterone Receptor Agonists

In some cases, the second therapeutic agent is a progesterone receptor agonist, such as Drospirenone. In some cases, a method of the present disclosure comprises administering: a) remdesivir; and b) a progesterone receptor agonist in combined effective amounts to an individual having an RNA virus infection, e.g., an individual having a disease caused by the RNA virus infection. In some cases, a method of the present disclosure comprises administering: a) remdesivir; and b) Drospirenone in combined effective amounts to an individual having an RNA virus infection, e.g., an individual having a disease caused by the RNA virus infection. In some cases, the individual is infected with SARS-CoV2. In some cases, the individual has Covid-19. Drospirenone has the following structure:

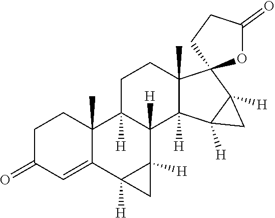

### Non-Steroidal Anti-Inflammatory Drugs

In some cases, the second therapeutic agent is a non-steroidal anti-inflammatory drug (NSAID). In some cases, the NSAID is a selective inhibitor of cyclooxygenase-2 (Cox-2). Cox-2 inhibitors include, e.g., celecoxib, rofecoxib, and valdecoxib.

In some cases, a method of the present disclosure comprises administering: a) remdesivir; and b) an NSAID in combined effective amounts to an individual having an RNA virus infection, e.g., an individual having a disease caused by the RNA virus infection. In some cases, a method of the present disclosure comprises administering: a) remdesivir; and b) a Cox-2 inhibitor in combined effective amounts to an individual having an RNA virus infection, e.g., an individual having a disease caused by the RNA virus infection. In some cases, a method of the present disclosure comprises administering: a) remdesivir; and b) a selective Cox-2 inhibitor in combined effective amounts to an individual having an RNA virus infection, e.g., an individual having a disease caused by the RNA virus infection. In some cases, a method of the present disclosure comprises administering: a) remdesivir; and b) valdecoxib in combined effective amounts to an individual having an RNA virus infection, e.g., an individual having a disease caused by the RNA virus infection. In some cases, the individual is infected with SARS-CoV2. In some cases, the individual has Covid-19. Valdecoxib has the following structure:

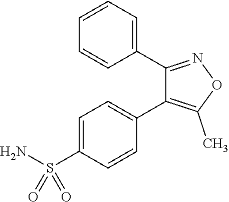 [SMILES: Cc1onc(-c2ccccc2)c1-c1ccc(S(N)(=O)=O)cc1]

### Sodium Glucose Co-Transporter-2 (SGLT-2) Inhibitors

In some cases, the second therapeutic agent is an SGLT-2 inhibitor. SGLT-2 inhibitors include, e.g., ertugliflozin, canagliflozin, empagliflozin, and dapagliflozin.

In some cases, a method of the present disclosure comprises administering: a) remdesivir; and b) an SGLT-2 inhibitor in combined effective amounts to an individual having an RNA virus infection, e.g., an individual having a disease caused by the RNA virus infection. In some cases, a method of the present disclosure comprises administering: a) remdesivir; and b) empagliflozin in combined effective amounts to an individual having an RNA virus infection, e.g., an individual having a disease caused by the RNA virus infection. In some cases, the individual is infected with SARS-CoV2. In some cases, the individual has Covid-19. Empagliflozin has the following structure:

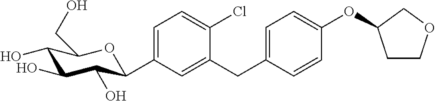

### Niemann-Pick C1-Like 1 (NPC1L1) Protein Blocker

In some cases, the second therapeutic agent is an agent that blocks the critical mediator of cholesterol absorption, the Niemann-Pick C1-like 1 (NPC1L1) protein. In some cases, the NPC1L1 inhibitor is ezetimibe.

In some cases, a method of the present disclosure comprises administering: a) remdesivir; and b) an NPC1L1 inhibitor in combined effective amounts to an individual having an RNA virus infection, e.g., an individual having a disease caused by the RNA virus infection. In some cases, a method of the present disclosure comprises administering: a) remdesivir; and b) ezetimibe in combined effective amounts to an individual having an RNA virus infection, e.g., an individual having a disease caused by the RNA virus infection. In some cases, the individual is infected with SARS-CoV2. In some cases, the individual has Covid-19. Ezetimibe has the following structure:

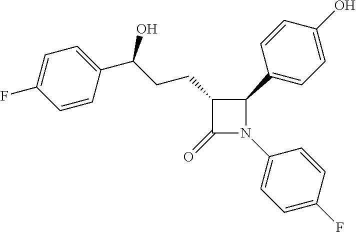 [SMILES: O=C1[C@H](CC[C@H](O)c2ccc(F)cc2)[C@@H](c2ccc(O)cc2)N1c1ccc(F)cc1]

### Isocitrate Dehydrogenase-1 (IDH1) Inhibitors

In some cases, the second therapeutic agent is an IDH1 inhibitor. In some cases, the IDH1 inhibitor is Ivosidenib.

In some cases, a method of the present disclosure comprises administering: a) remdesivir; and b) an IDH1 inhibitor in combined effective amounts to an individual having an RNA virus infection, e.g., an individual having a disease caused by the RNA virus infection. In some cases, a method of the present disclosure comprises administering: a) remdesivir; and b) Ivosidenib in combined effective amounts to an individual having an RNA virus infection, e.g., an individual having a disease caused by the RNA virus infection. In some cases, the individual is infected with SARS-CoV2. In some cases, the individual has Covid-19. Ivosidenib has the following structure:

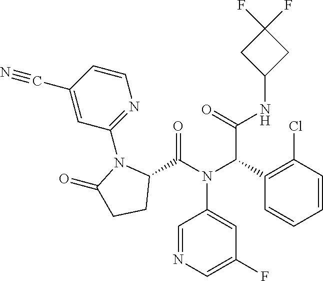 [SMILES: N#Cc1ccnc(N2C(=O)CC[C@H]2C(=O)N(c2cncc(F)c2)[C@H](C(=O)NC2CC(F)(F)C2)c2ccccc2Cl)c1]

### Protein Folding Chaperones

In some cases, the second therapeutic agent is a protein folding chaperone. In some cases, the protein folding chaperone is Lumacaftor.

In some cases, a method of the present disclosure comprises administering: a) remdesivir; and b) a protein folding chaperone in combined effective amounts to an individual having an RNA virus infection, e.g., an individual having a disease caused by the RNA virus infection. In some cases, a method of the present disclosure comprises administering: a) remdesivir; and b) Lumacaftor in combined effective amounts to an individual having an RNA virus infection, e.g., an individual having a disease caused by the RNA virus infection. In some cases, the individual is infected with SARS-CoV2. In some cases, the individual has Covid-19. Lumacaftor has the following structure:

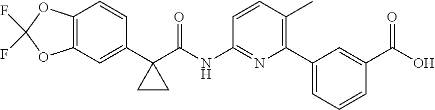 [SMILES: Cc1ccc(NC(=O)C2(c3ccc4c(c3)OC(F)(F)O4)CC2)nc1-c1cccc(C(=O)O)c1]

### P2Y₁₂ ADP Receptor Antagonists

In some cases, the second therapeutic agent is a P2Y₁₂ adenosine diphosphate (ADP) receptor antagonist. P2Y₁₂ ADP receptor antagonists include, e.g., clopidogrel, ticlopidine, ticagrelor, prasugrel, and cangrelor. In some cases, the P2Y₁₂ ADP receptor antagonist is prasugrel.

In some cases, a method of the present disclosure comprises administering: a) remdesivir; and b) a P2Y₁₂ ADP receptor antagonist in combined effective amounts to an individual having an RNA virus infection, e.g., an individual having a disease caused by the RNA virus infection. In some cases, a method of the present disclosure comprises administering: a) remdesivir; and b) prasugrel in combined effective amounts to an individual having an RNA virus infection, e.g., an individual having a disease caused by the RNA virus infection. In some cases, the individual is infected with SARS-CoV2. In some cases, the individual has Covid-19. Prasugrel has the following structure:

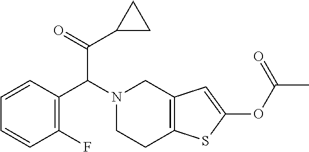 [SMILES: CC(=O)Oc1cc2c(s1)CCN(C(C(=O)C1CC1)c1ccccc1F)C2]

### Angiotensin Converting Enzyme (ACE) Inhibitor

In some cases, the second therapeutic agent is an ACE inhibitor. ACE inhibitors include, e.g., benazepril, captopril, enalapril, fosinopril, lisinopril, moexipril, perindopril, and quinapril.

In some cases, a method of the present disclosure comprises administering: a) remdesivir; and b) an ACE inhibitor in combined effective amounts to an individual having an RNA virus infection, e.g., an individual having a disease caused by the RNA virus infection. In some cases, a method of the present disclosure comprises administering: a) remdesivir; and b) quinapril (e.g., quinapril hydrochloride) in combined effective amounts to an individual having an RNA virus infection, e.g., an individual having a disease caused by the RNA virus infection. In some cases, the individual is infected with SARS-CoV2. In some cases, the individual has Covid-19. Quinapril hydrochloride has the following structure:

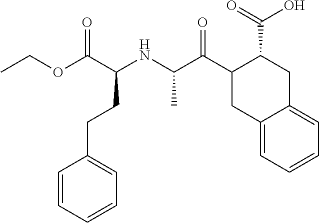 [SMILES: CCOC(=O)[C@H](CCc1ccccc1)N[C@@H](C)C(=O)C1Cc2ccccc2C[C@H]1C(=O)O]

### Rifamycin Antibiotics

In some cases, a method of the present disclosure comprises administering: a) remdesivir; and b) a rifamycin antibiotic in combined effective amounts to an individual having an RNA virus infection, e.g., an individual having a disease caused by the RNA virus infection. In some cases, a method of the present disclosure comprises administering: a) remdesivir; and b) rifamycin in combined effective amounts to an individual having an RNA virus infection, e.g., an individual having a disease caused by the RNA virus infection. In some cases, the individual is infected with SARS-CoV2. In some cases, the individual has Covid-19.

In some cases, a method of the present disclosure comprises administering: a) remdesivir; and b) three additional therapeutic agents, where the three additional therapeutic agents are sofosbuvir, velpatasvir, and etravirine, in combined effective amounts to an individual having an RNA virus infection, e.g., an individual having a disease caused by the RNA virus infection. In some cases, the individual is infected with SARS-CoV2. In some cases, the individual has Covid-19. Etravirine has the following structure:

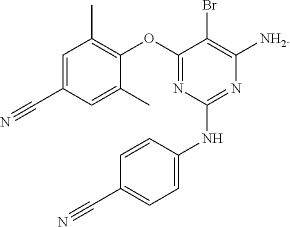 [SMILES: Cc1cc(C#N)cc(C)c1Oc1nc(Nc2ccc(C#N)cc2)nc(N)c1Br]

In some cases, a method of the present disclosure comprises administering: a) remdesivir; and b) three additional therapeutic agents, where the three additional therapeutic agents are Sofosbuvir, velpatasvir, and grazoprevir, in combined effective amounts to an individual having an RNA virus infection, e.g., an individual having a disease caused by the RNA virus infection. In some cases, the individual is infected with SARS-CoV2. In some cases, the individual has Covid-19.

In some cases, a method of the present disclosure comprises administering: a) remdesivir; and b) three additional therapeutic agents, where the three additional therapeutic agents are Sofosbuvir, velpatasvir, and favipiravir in combined effective amounts to an individual having an RNA virus infection, e.g., an individual having a disease caused by the RNA virus infection. In some cases, the individual is infected with SARS-CoV2. In some cases, the individual has Covid-19. Favipiravir has the following structure:

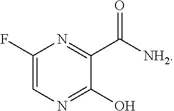 [SMILES: NC(=O)c1nc(F)cnc1O]

In some cases, a method of the present disclosure comprises administering: a) remdesivir; and b) three additional therapeutic agents, where the three additional therapeutic agents are Sofosbuvir, velpatasvir, ebselen, in combined effective amounts to an individual having an RNA virus infection, e.g., an individual having a disease caused by the RNA virus infection. In some cases, the individual is infected with SARS-CoV2. In some cases, the individual has Covid-19. Ebselen has the following structure:

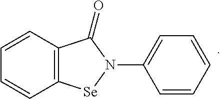 [SMILES: O=c1c2ccccc2[se]n1-c1ccccc1]

In some cases, a method of the present disclosure comprises administering: a) remdesivir; and b) daclatasvir dihydrochloride in combined effective amounts to an individual having an RNA virus infection, e.g., an individual having a disease caused by the RNA virus infection. In some cases, the individual is infected with SARS-CoV2. In some cases, the individual has Covid-19. Daclatasvir dihydrochloride has the following structure:

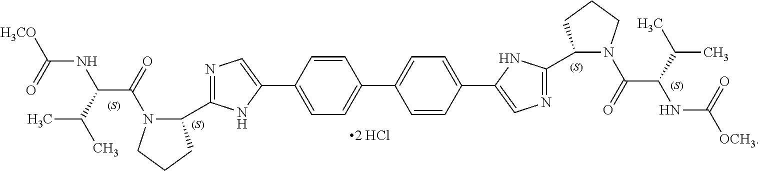

In some cases, a method of the present disclosure comprises administering: a) remdesivir; and b) ledipasvir in combined effective amounts to an individual having an RNA virus infection, e.g., an individual having a disease caused by the RNA virus infection. In some cases, the individual is infected with SARS-CoV2. In some cases, the individual has Covid-19. Ledipasvir has the following structure:

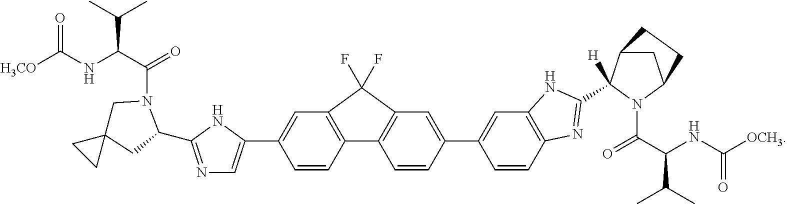 [SMILES: COC(=O)N[C@H](C(=O)N1CC2(CC2)C[C@H]1c1ncc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)[nH]1)C(C)C]

In some cases, a method of the present disclosure comprises administering: a) remdesivir; and b) mycophenolic acid in combined effective amounts to an individual having an RNA virus infection, e.g., an individual having a disease caused by the RNA virus infection. In some cases, the individual is infected with SARS-CoV2. In some cases, the individual has Covid-19. Mycophenolic acid has the following structure:

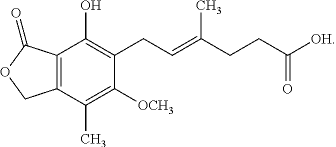 [SMILES: COc1c(C)c2c(c(O)c1C/C=C(\C)CCC(=O)O)C(=O)OC2]

In some cases, a method of the present disclosure comprises administering: a) remdesivir; and b) pibrentasvir (ABT-530) in combined effective amounts to an individual having an RNA virus infection, e.g., an individual having a disease caused by the RNA virus infection. In some cases, the individual is infected with SARS-CoV2. In some cases, the individual has Covid-19. Pibrentasvir acid has the following structure:

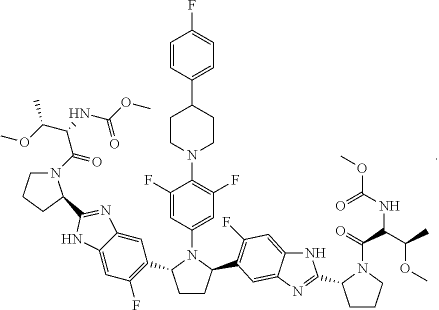

In some cases, a method of the present disclosure comprises administering: a) remdesivir; and b) imatinib in combined effective amounts to an individual having an RNA virus infection, e.g., an individual having a disease caused by the RNA virus infection. In some cases, the individual is infected with SARS-CoV2. In some cases, the individual has Covid-19. Imatinib acid has the following structure:

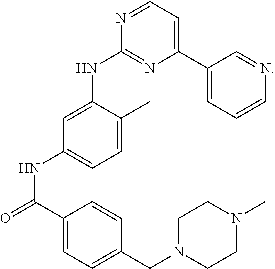 [SMILES: Cc1ccc(NC(=O)c2ccc(CN3CCN(C)CC3)cc2)cc1Nc1nccc(-c2cccnc2)n1]

In some cases, a method of the present disclosure comprises administering: a) remdesivir; and b) ledipasvir acetone in combined effective amounts to an individual having an RNA virus infection, e.g., an individual having a disease caused by the RNA virus infection. In some cases, the individual is infected with SARS-CoV2. In some cases, the individual has Covid-19. Ledipasvir acetone acid has the following structure:

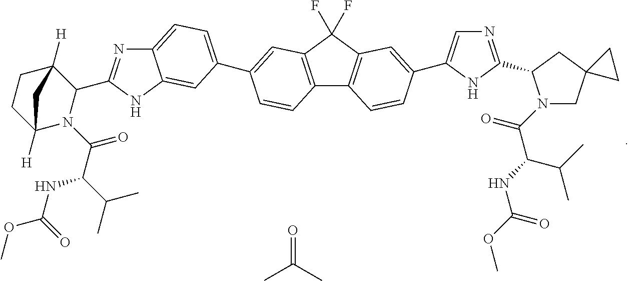

In some cases, a method of the present disclosure comprises administering: a) remdesivir; and b) ombitasvir (ABT-267) in combined effective amounts to an individual having an RNA virus infection, e.g., an individual having a disease caused by the RNA virus infection. In some cases, the individual is infected with SARS-CoV2. In some cases, the individual has Covid-19. Ombitasvir acid has the following structure:

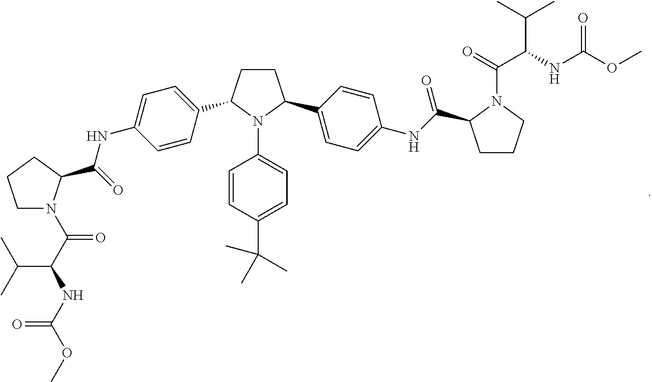 [SMILES: COC(=O)N[C@H](C(=O)N1CCC[C@H]1C(=O)Nc1ccc([C@@H]2CC[C@@H](c3ccc(NC(=O)[C@@H]4CCCN4C(=O)[C@@H](NC(=O)OC)C(C)C)cc3)N2c2ccc(C(C)(C)C)cc2)cc1)C(C)C]

### Additional Agents

A method of the present disclosure can comprise, in addition to administering an RdRp such as remdesivir and a second therapeutic agent, as described above, administering one or more further therapeutic agents, e.g., therapeutic agents suitable for treating Covid-19 or for treating an individual who has been infected with SARS-CoV2 or is at high risk of being infected with SARS-CoV2. In some cases, the further therapeutic agent suitable for treating Covid-19 is an antibody specific for the spike glycoprotein of SARS-CoV2. For example, the antibody can be a neutralizing antibody that binds specifically to the spike glycoprotein of SARS-CoV2. Suitable antibodies include single-chain Fv antibodies, nanobodies, IgG antibodies, and the like. In some cases, two or more antibodies specific for the spike glycoprotein of SARS-CoV2 are administered. In some cases, the further therapeutic agent is famotidine. In some cases, the further therapeutic agent suitable for treating Covid-19 is dexamethasone.

### Dosages and Routes of Administration

A suitable dosage of a first therapeutic agent (e.g., an RdRp inhibitor, such as remdesivir) and a second therapeutic agent, as described above, can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A first therapeutic agent and/or a second therapeutic agent, as described above, may be administered in amounts between 1 ng/kg body weight and 20 mg/kg body weight per dose, e.g. between 0.1 mg/kg body weight to 10 mg/kg body weight, e.g. between 0.5 mg/kg body weight to 5 mg/kg body weight; however, doses below or above this exemplary range are envisioned, especially considering the aforementioned factors. If the regimen is a continuous infusion, it can also be in the range of 1 μg to 10 mg per kilogram of body weight per minute. A first therapeutic agent and/or a second therapeutic agent, as described above, can be administered in an amount of from about 1 mg/kg body weight to 50 mg/kg body weight, e.g., from about 1 mg/kg body weight to about 5 mg/kg body weight, from about 5 mg/kg body weight to about 10 mg/kg body weight, from about 10 mg/kg body weight to about 15 mg/kg body weight, from about 15 mg/kg body weight to about 20 mg/kg body weight, from about 20 mg/kg body weight to about 25 mg/kg body weight, from about 25 mg/kg body weight to about 30 mg/kg body weight, from about 30 mg/kg body weight to about 35 mg/kg body weight, from about 35 mg/kg body weight to about 40 mg/kg body weight, or from about 40 mg/kg body weight to about 50 mg/kg body weight.

In some cases, a suitable dose of a first therapeutic agent and/or a second therapeutic agent, as described above, is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight. Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a first therapeutic agent and/or a second therapeutic agent, as described above, is administered in maintenance doses, ranging from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight.

In some cases, a “loading dose” of remdesivir is administered on Day 1; and a maintenance dose is administered starting on Day 2. In some cases, the maintenance dose of remdesivir is administered once daily.

In some cases, multiple doses of a first therapeutic agent and/or a second therapeutic agent, as described above, are administered. The frequency of administration of a first therapeutic agent and/or a second therapeutic agent can vary depending on any of a variety of factors, e.g., severity of the symptoms, etc. For example, in some cases, a first therapeutic agent and/or a second therapeutic agent, as described above, is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The duration of administration of a first therapeutic agent and/or a second therapeutic agent, as described above, e.g., the period of time over which the first and/or the second therapeutic agent is administered, can vary, depending on any of a variety of factors, e.g., patient response, etc. For example, a first therapeutic agent and/or a second therapeutic agent, as described above, can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

An active agent (a first therapeutic agent and/or a second therapeutic agent, as described above) is administered to an individual using any available method and route suitable for drug delivery, including in vivo and ex vivo methods, as well as systemic and localized routes of administration.

Conventional and pharmaceutically acceptable routes of administration include intramuscular, intratracheal, intralymphatic, intracranial, subcutaneous, intradermal, topical application, intravenous, intraarterial, rectal, nasal, oral, and other enteral and parenteral routes of administration. Suitable routes of administration include pulmonary administration such as in a nebulized or other aerosolized form. Routes of administration may be combined, if desired, or adjusted depending upon the therapeutic agent and/or the desired effect. A first therapeutic agent and/or a second therapeutic agent, as described above can be administered in a single dose or in multiple doses.

A method of the present disclosure comprises administering: i) a first therapeutic agent, e.g., an RdRp inhibitor such as remdesivir; and ii) at least a second therapeutic agent, as described above. The first therapeutic agent and the second therapeutic agent can be administered at substantially the same time or at different times. In some cases, an RdRp inhibitor such as remdesivir is administered via oral administration. In some cases, an RdRp inhibitor such as remdesivir is administered via intravenous administration. In some cases, an RdRp inhibitor such as remdesivir is administered via inhalation. In some cases, an RdRp inhibitor such as remdesivir is administered via a pulmonary route.

The administration of the first therapeutic agent and the second therapeutic agent can be substantially simultaneous, e.g., the first therapeutic agent can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the second therapeutic agent. In some cases, first therapeutic agent is administered to an individual who is undergoing treatment with the second therapeutic agent. The administration of the first therapeutic agent and the second therapeutic agent can occur at different times and/or at different frequencies.

As another example, a treatment method of the present disclosure comprises co-administration of a first therapeutic agent and a second therapeutic agent. By “co-administration” is meant that both a first therapeutic agent and a second therapeutic agent are administered to an individual, although not necessarily at the same time, in order to achieve a therapeutic effect that is the result of having administered both the first therapeutic agent and the second therapeutic agent. The administration of the first therapeutic agent and the second therapeutic agent can be substantially simultaneous, e.g., the first therapeutic agent can be administered to an individual within about 1 minute to about 24 hours (e.g., within about 1 minute, within about 5 minutes, within about 15 minutes, within about 30 minutes, within about 1 hour, within about 4 hours, within about 8 hours, within about 12 hours, or within about 24 hours) of administration of the second therapeutic agent. In some cases, the first therapeutic agent is administered to an individual who is undergoing treatment with the second therapeutic agent. In some cases, the first therapeutic agent and the second therapeutic agent are administered to an individual who has not previously been treated with the second therapeutic agent. The administration of the first therapeutic agent and the second therapeutic agent can occur at different times and/or at different frequencies.

### RNA Viruses

A method of the present disclosure is suitable for treating a disease caused by a viral infection, e.g., an RNA virus infection. A method of the present disclosure is suitable for treating an individual who has been infected with an RNA virus, or who is at high risk of being infected with an RNA virus, or who is at high risk of developing a disease due to infection with an RNA virus.

RNA viruses include, e.g., Ebola virus, West Nile virus, Hepatitis C virus, Poliovirus, measles virus, influenza virus, rhinovirus, SARS-CoV-1, SARS-CoV2, MERS, and the like. In some cases, the RNA virus is SARS-CoV-2 (Severe acute respiratory syndrome coronavirus 2).

A method of the present disclosure is suitable for treating a disease caused by SARS-CoV-2, including a variant of SARS-CoV-2. For example, a method of the present disclosure is suitable for treating a disease caused by the WA1 variant of SARS-CoV-2. As another example, a method of the present disclosure is suitable for treating a disease caused by the P.1 variant of SARS-CoV-2. As another example, a method of the present disclosure is suitable for treating a disease caused by the B.1.429 variant of SARS-CoV-2. As another example, a method of the present disclosure is suitable for treating a disease caused by the B.1.351 variant of SARS-CoV-2. As another example, a method of the present disclosure is suitable for treating a disease caused by the B.1.1.7 variant of SARS-CoV-2. As another example, a method of the present disclosure is suitable for treating a disease caused by the B.1.617.2 variant of SARS-CoV-2.

### Individuals Suitable for Treatment

Individuals suitable for treatment with a method of the present disclosure include: i) individuals infected with an RNA virus; ii) individuals at high risk of being infected with an RNA virus; iii) individuals at high risk of developing a disease due to infection with an RNA virus; and iv) individuals having a disease due to infection with an RNA virus.

Individuals suitable for treatment with a method of the present disclosure include: i) individuals infected with SARS-CoV-2; ii) individuals at high risk of being infected with SARS-CoV2; iii) individuals at high risk of developing Covid-19 (disease caused by infection with SARS-CoV2); and iv) individuals having Covid-19. In some cases, the individual has been diagnosed as having a SARS-CoV2 infection. In some cases, the individual has been diagnosed as having a SARS-CoV2 infection and exhibits one or more symptoms of Covid-19.

In some cases, the individual is at higher risk than the general population of becoming infected with SARS-CoV2. Such individuals include medical personnel who come into contact (e.g., frequent contact) with Covid-19 patients. Such individuals include first responders who come into contact (e.g., frequent contact) with Covid-19 patients.

In some cases, the individual has Covid-19. In some cases, the individual exhibits one or more symptoms of a SARS-CoV2 infection, e.g., one or more of fever, fatigue, cough, and difficulty breathing. In some cases, the individual is infected with SARS-CoV2 and has an oxygen saturation of less than 94%. In some cases, the individual is infected with SARS-CoV2 and is receiving supplemental oxygen. In some cases, the individual is infected with SARS-CoV2 and requires mechanical ventilation or extracorporeal membrane oxygenation. In some cases, the individual is 60 years old or older; e.g., in some cases, the individual is from 60 years old to 65 years old, from 65 years old to 70 years old, from 70 years old to 75 years old, from 75 years old to 80 years old, from 80 years old to 85 years old, from 85 years old to 90 years old, or older than 90 years. In some cases, the individual is from 20 years old to 30 years old. In some cases, the individual is from 30 years old to 40 years old. In some cases, the individual is from 40 years old to 50 years old. In some cases, the individual is from 50 years old to 60 years old. In some cases, the individual is a teenager. In some cases, the individual is an adolescent. In some cases, the individual is a pediatric patient. In some cases, the individual is from 5 years old to 10 years old. In some cases, the individual is from 10 years old to 12 years old. In some cases, the individual is from 1 year old to 5 years old. In some cases, the individual an infant.

In some cases, the individual is one who has not previously been treated with the second therapeutic agent.

### EXAMPLES OF NON-LIMITING ASPECTS OF THE DISCLOSURE

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A method of treating a viral infection caused by an RNA virus, the method comprising administering a first therapeutic agent and a second therapeutic agent (at least one additional therapeutic agent) in combined effective amounts, wherein: a) the first therapeutic agent is an inhibitor of an RNA-dependent RNA polymerase (RdRp); and b) the second therapeutic agent is an agent other than an RdRp inhibitor.
Aspect 2. The method of aspect 1, wherein the second therapeutic agent (the at least one additional therapeutic agent) is selected from: a Hepatitis C Virus (HCV) inhibitor, an inhibitor of B-Raf, a proton pump inhibitor (PPI), an angiotensin IT receptor blocker or antagonist, a prostacyclin receptor agonist, a calcium channel blocker, a dihydropyridine-type calcium channel blocker, a leukotriene receptor antagonist, a retinoid that selectively activates a retinoid X receptor, a corticosteroid, a selective inhibitor of phosphodiesterase type 3, an arginine vasopressin (AVP) receptor antagonist, an agonist of the progesterone receptor (PR), a selective inhibitor of cyclooxygenase-2, an inhibitor of the sodium glucose co-transporter-2 (SGLT-2), a Niemann-Pick C1-like 1 (NPC1L1) protein blocker, an inhibitor of isocitrate dehydrogenase-1 (IDH1), a protein folding chaperone, a corticosteroid, an antagonist of P2Y₁₂ adenosine diphosphate (ADP) receptor, and an angiotensin converting enzyme (ACE) inhibitor; or wherein the at least one additional therapeutic agent is selected from: i) Sofosbuvir, velpatasvir, and grazoprevir; ii) Sofosbuvir, velpatasvir, and etravirine; iii) Sofosbuvir, velpatasvir, and favipiravir; iv) Sofosbuvir, velpatasvir, and ebselen; v) Daclatasvir dihydrochloride; vi) Ledipasvir; vii) mycophenolic acid; viii) ABT-530 (Pibrentasvir); ix) Imatinib; x) Ledipasvir acetone; and xi) ABT-267 (Ombitasvir).
Aspect 3. The method of aspect 1 or aspect 2, wherein the first therapeutic agent and the second therapeutic agent (the at least one additional therapeutic agent) are administered in synergistically effective amounts.
Aspect 4. The method of aspect 2, wherein the HCV inhibitor is Velpatasvir.
Aspect 5. The method of aspect 1, wherein the HCV inhibitor is Elbasvir.
Aspect 6. The method of aspect 5, further comprising administering Grazoprevir, boceprevir, simeprevir, or elaprevir.
Aspect 7. The method of aspect 4 or aspect 5, further comprising administering Sofosbuvir.
Aspect 8. The method of aspect 1, wherein the inhibitor of B-Raf is Dabrafenib.
Aspect 9. The method of aspect 1, wherein the PPI is Omeprazole.
Aspect 10. The method of aspect 1, wherein the angiotensin II receptor blocker is Telmisartan.
Aspect 11. The method of aspect 1, wherein the angiotensin II receptor antagonist is Irbesartan.
Aspect 12. The method of aspect 1, wherein the prostacyclin receptor agonist is Selexipag.
Aspect 13. The method of aspect 1, wherein the calcium channel blocker is Nifedipine or Nimodipine.
Aspect 14. The method of aspect 1, wherein the leukotriene receptor antagonist is Zafirlukast.
Aspect 15. The method of aspect 1, wherein the retinoid is Bexarotene.
Aspect 16. The method of aspect 1, wherein the PDE₃ inhibitor is Cilostazol.
Aspect 17. The method of aspect 1, wherein the AVP receptor antagonist is Conivaptan hydrochloride.
Aspect 18. The method of aspect 1, wherein the PR agonist is Drospirenone.
Aspect 19. The method of aspect 1, wherein the selective inhibitor of cyclooxygenase-2 is Valdecoxib.
Aspect 20. The method of aspect 1, wherein the SGLT-2 inhibitor is Empagliflozin.
Aspect 21. The method of aspect 1, wherein the NPC1L1 blocker is Ezetimibe.
Aspect 22. The method of aspect 1, wherein the IDH1 inhibitor is Ivosidenib.
Aspect 23. The method of aspect 1, wherein the protein folding chaperone is Lumacaftor.
Aspect 24. The method of aspect 1, wherein the corticosteroid is Meprednisone, Methylprednisolone, Budesonide, or Clobetasol propionate.
Aspect 25. The method of aspect 1, wherein the P2Y₁₂ ADP receptor antagonist is Prasugrel.
Aspect 26. The method of aspect 1, wherein the ACE inhibitor is Quinapril hydrochloride.
Aspect 27. The method of any one of aspects 1-26, wherein the RdRp inhibitor is sofosbuvir, remdesivir, ribavirin, favipiravir, pimodivir, or baloxavir.
Aspect 28. The method of any one of aspects 1-26, wherein the RdRp inhibitor is remdesivir.
Aspect 29. The method of any one of aspects 1-27, wherein the virus is SARS-CoV2.
Aspect 30. The method of aspect 29, further comprising administering an antibody specific for the SARS-CoV2 spike glycoprotein.
Aspect 31. The method of aspect 30, further comprising administering famotidine.
Aspect 32. The method of any one of aspects 29-31, wherein the individual has been diagnosed as having Covid-19.
Aspect 33. The method of any one of aspects 29-31, wherein the individual exhibits one or more symptoms of a SARS-CoV2 infection.
Aspect 34. The method of any one of aspects 29-33, wherein the individual has an oxygen saturation of less than 94%.
Aspect 35. The method of any one of aspects 29-34, wherein the individual is receiving supplemental oxygen.
Aspect 36. The method of any one of aspects 29-34, wherein the individual requires mechanical ventilation or extracorporeal membrane oxygenation.
Aspect 37. The method of any one of aspects 29-36, wherein the individual weighs 40 kg or more, and remdesivir is administered in a single loading dose of 200 mg on Day 1 followed by a once-daily maintenance dose of 100 mg from Day 2; or wherein remdesivir is administered in an amount that is at least 10%, at least 20%, at least 30%, at least 40%, at least 50%, at least 60%, at least 70%, at least 80%, or at least 90%, lower than a single loading dose of 200 mg on Day 1 followed by a once-daily maintenance dose of 100 mg from Day 2.
Aspect 38. The method of any one of aspects 29-36, wherein the individual weighs 3.5 kg to 40 kg, and remdesivir is administered in a single loading dose of 5 mg/kg on Day 1, followed by a once daily dose of 2.5 mg/kg from Day 2; or wherein remdesivir is administered in an amount that is at least 10%, at least 20%, at least 30%, at least 40%, at least 50%, at least 60%, at least 70%, at least 80%, or at least 90%, lower than a single loading dose of 5 mg/kg on Day 1, followed by a once daily dose of 2.5 mg/kg from Day 2.
Aspect 39. The method of aspect 37 or aspect 38, wherein the dose of remdesivir is administered orally.
Aspect 40. The method of aspect 37 or aspect 38, wherein the remdesivir is administered intravenously.
Aspect 41. The method of any one of aspects 29-40, wherein the SARS-CoV2 is a variant SARS-CoV2 selected from the B.1.1.7 variant, the B.1.351 variant, the B.1.617.2 variant, and the P.1 variant.
Aspect 42. The method of any one of aspects 29-41, wherein the RdRP inhibitor is remdesivir, and wherein the method comprises administering combined effective amounts of remdesivir, velpatasvir, and grazoprevir.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1

A high-throughput screen of a library of Food and Drug Administration (FDA) and European Medicines Agency (EMA) approved compounds (˜1200 small molecules) in a background of remdesivir was conducted using an in vitro cell-based assay. The assay was conducted in Vero-E6 cells and human lung epithelial cells (Calu-3) infected with SARS-CoV2, in presence of a ˜20% efficacious concentration of remdesivir empirically determined for each cell line, and varying FDA/EMA approved drugs at 40 μM final concentration. Activity of compounds was determined by inhibition of cytopathic effect (CPE; caused by the virus), via a luminescence assay measuring cell viability. Compounds that reproducibly increased remdesivir efficacy in both Vero-E6 and Calu-3 cells were prioritized and analyzed by a “checkerboard analysis”, comprehensively testing effect of various combinations of both agents at different concentrations. Synergy is an effect significantly greater than just the sum of the effects of the individual drugs. This data can be mathematically analyzed by the synergyfinder package in R (Ianevski et al., (2017) Bioinformatics 33, 2413) to differentiate additive from synergistic effects. The BLISS independence model, which assumes a stochastic process in which two drugs elicit their effects independently, was used. The expected combination effect was calculated based on the probability of independent events.

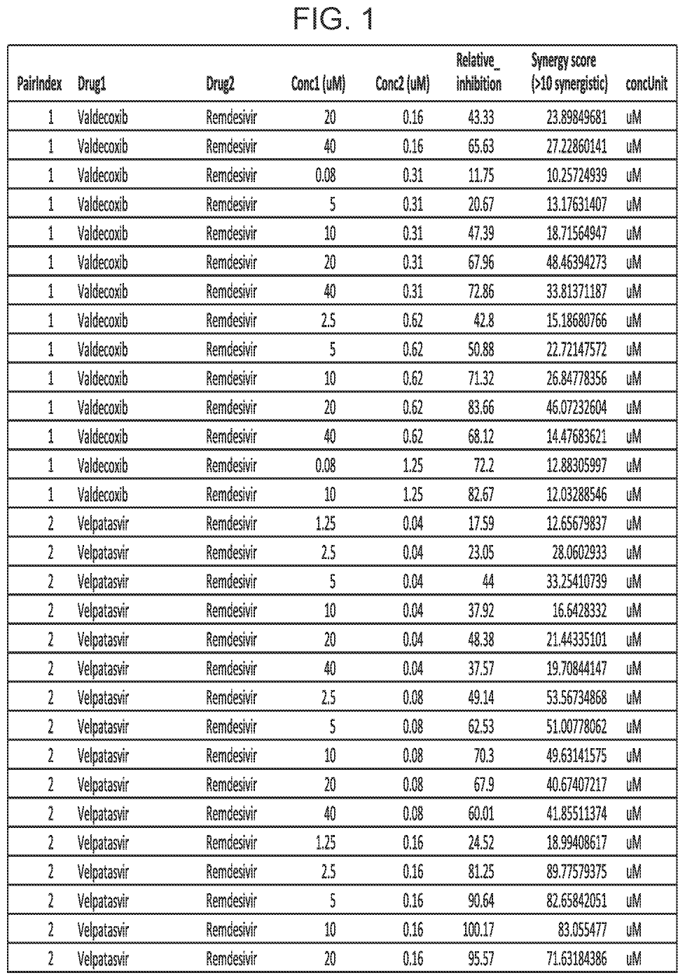

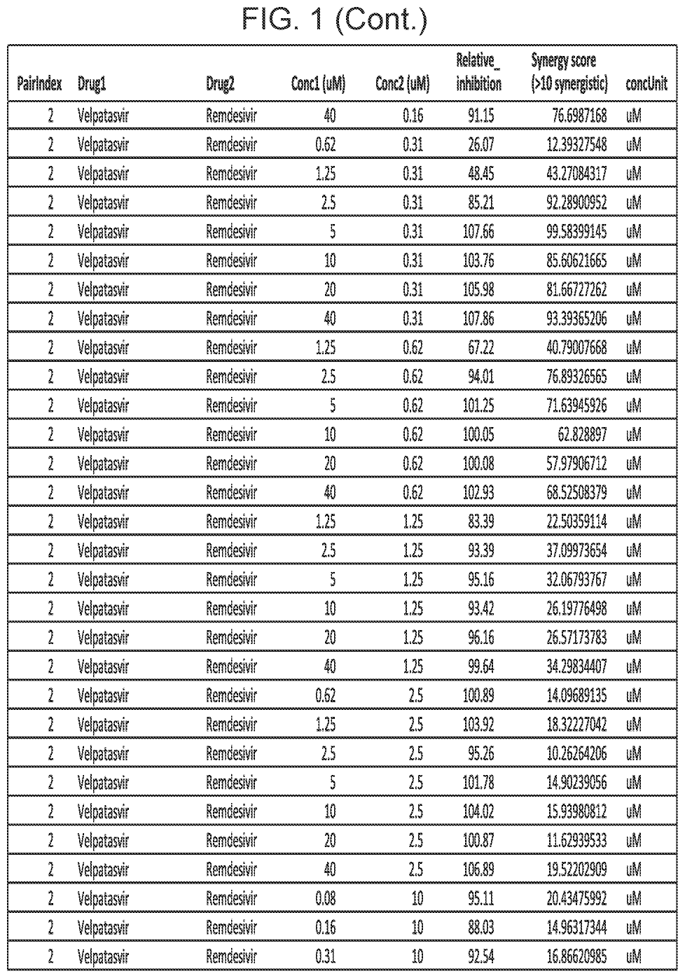

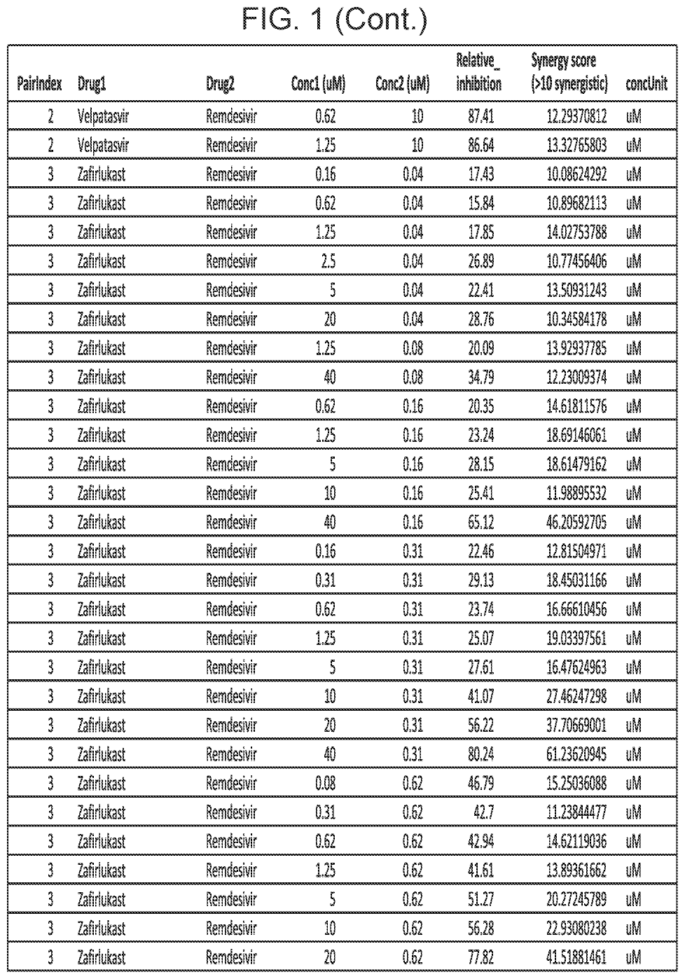

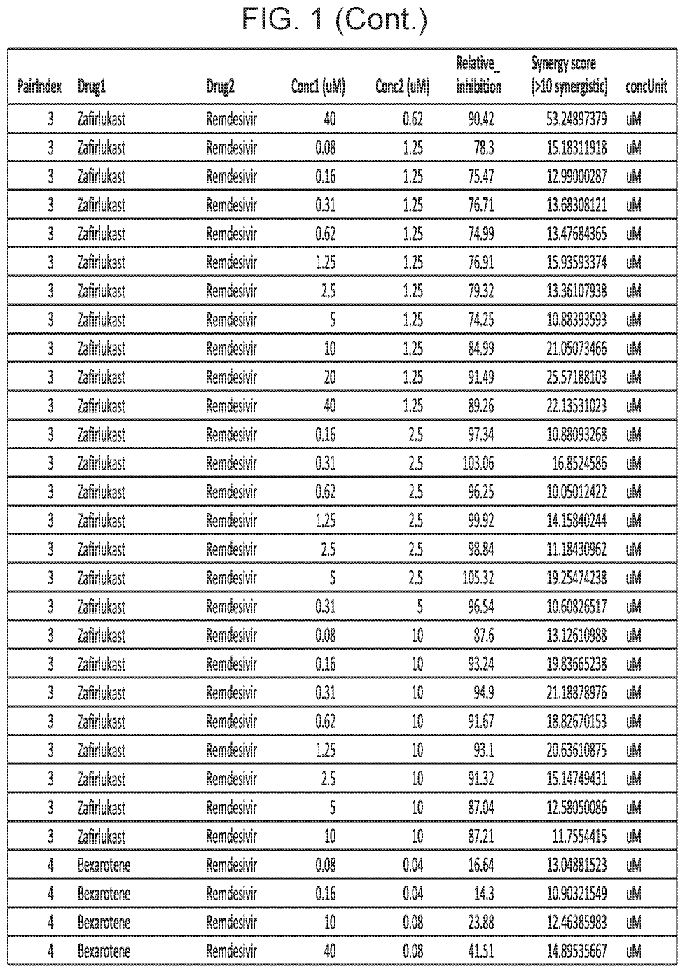

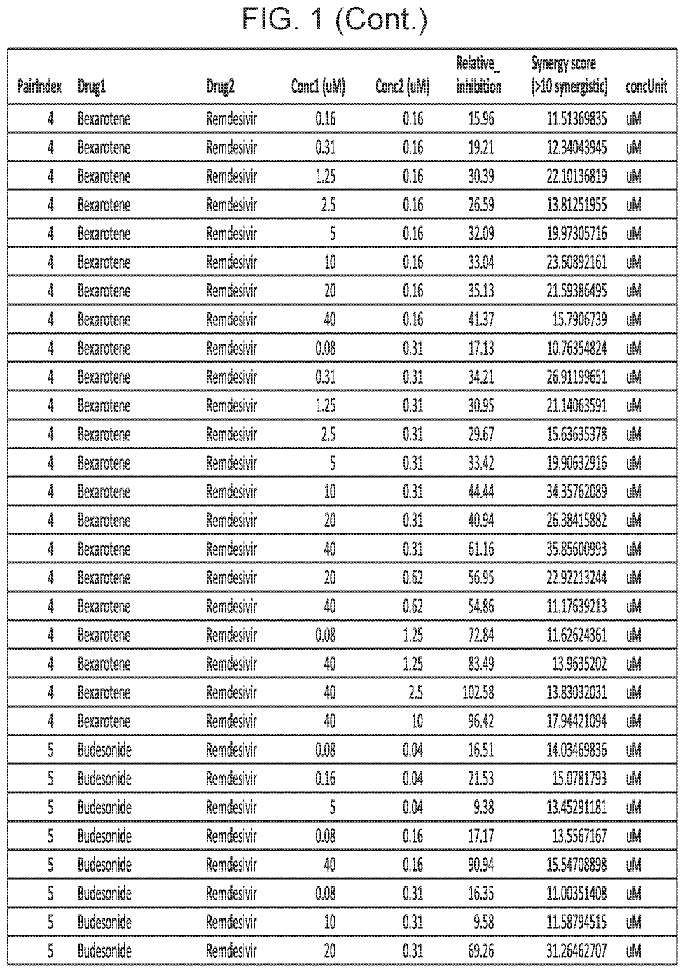

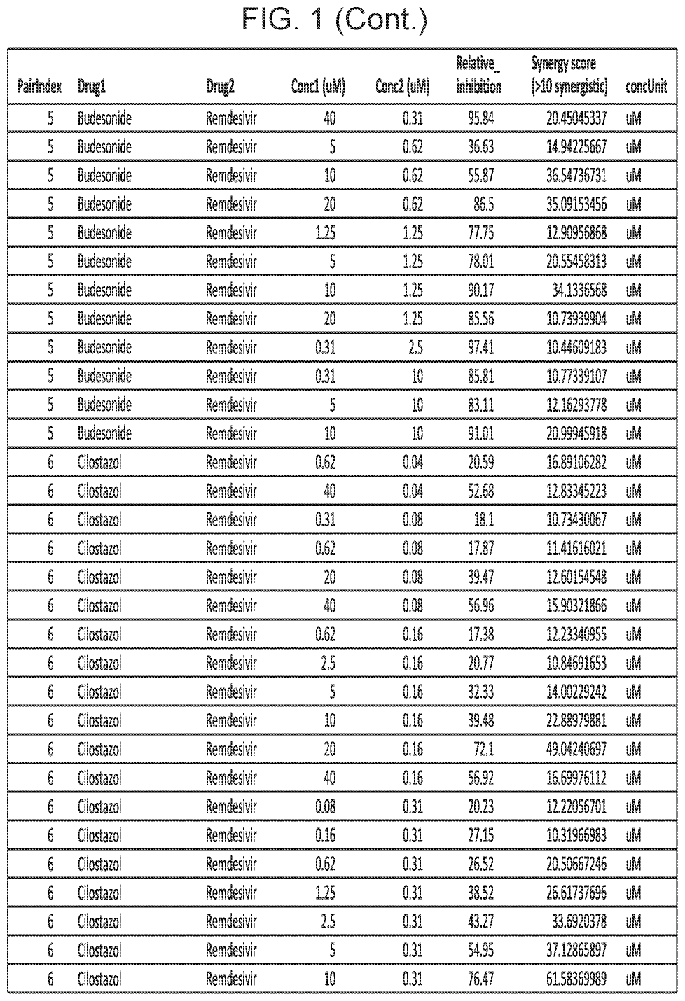

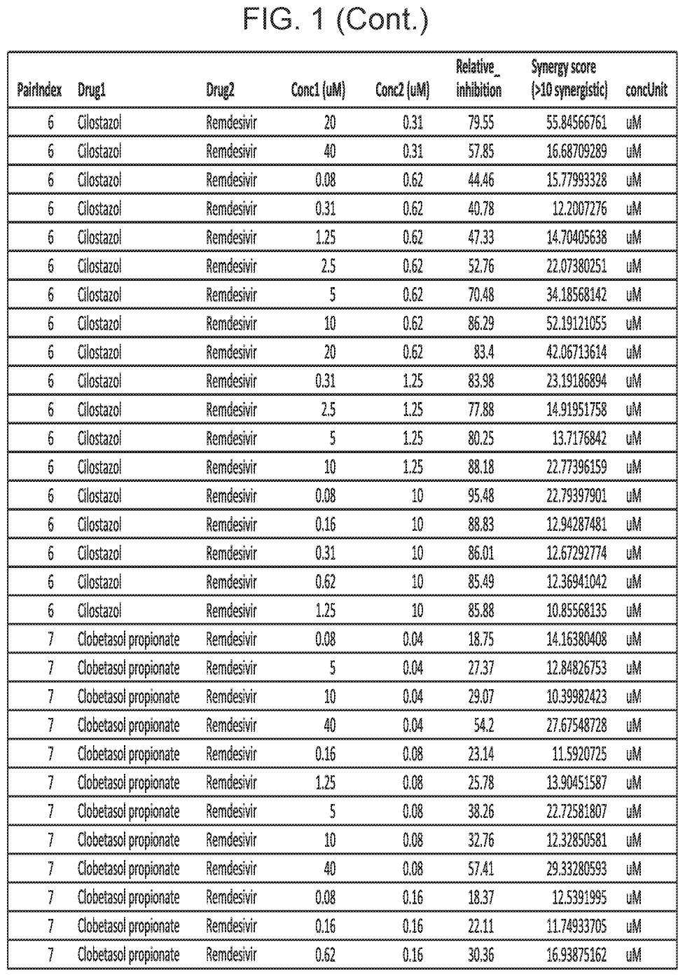

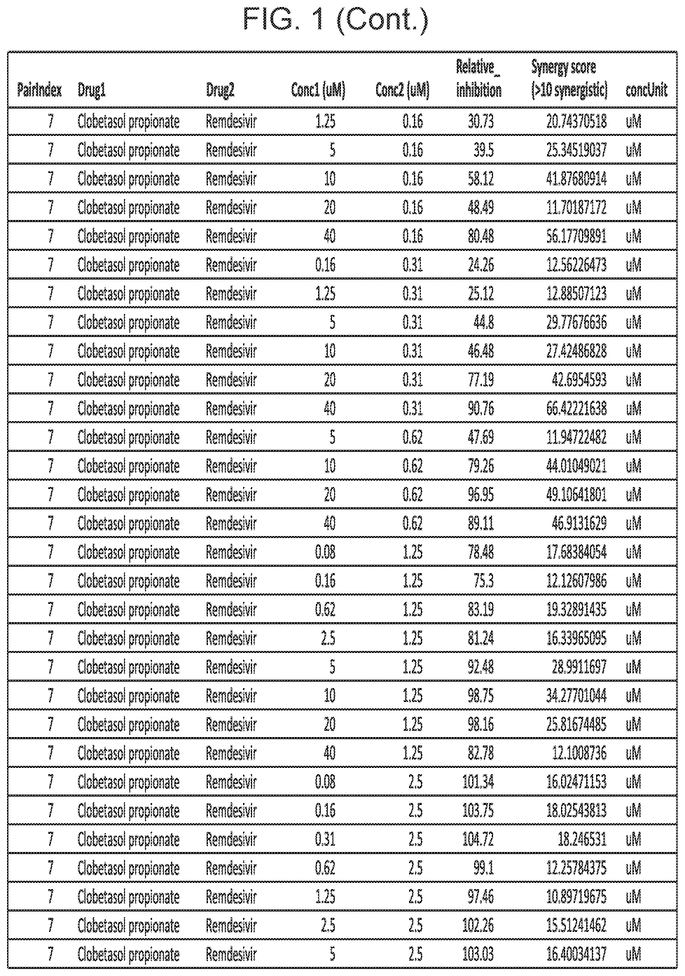

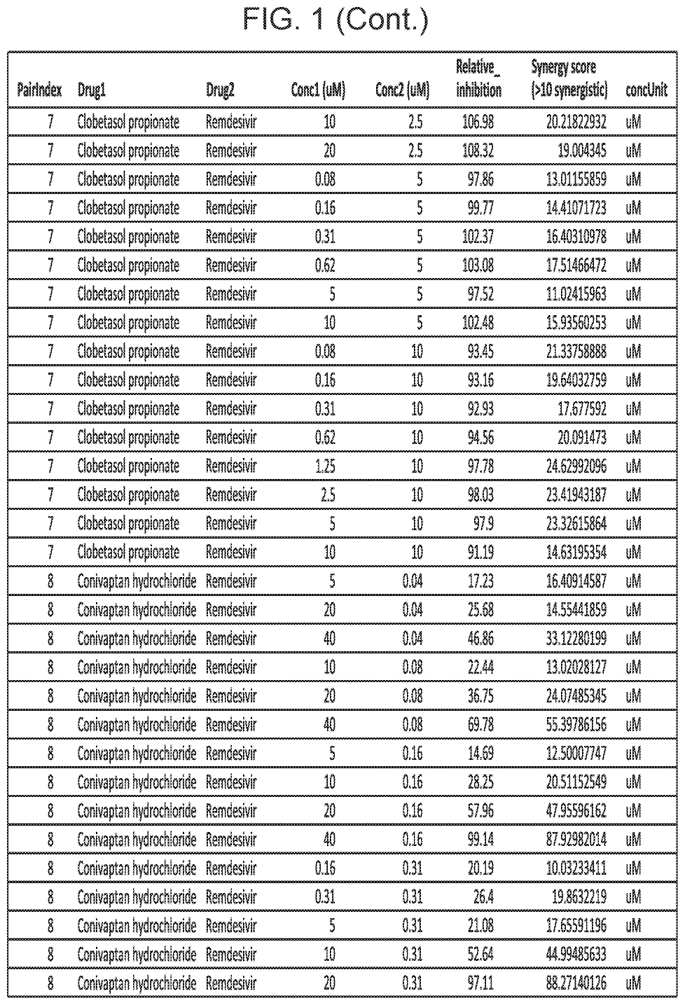

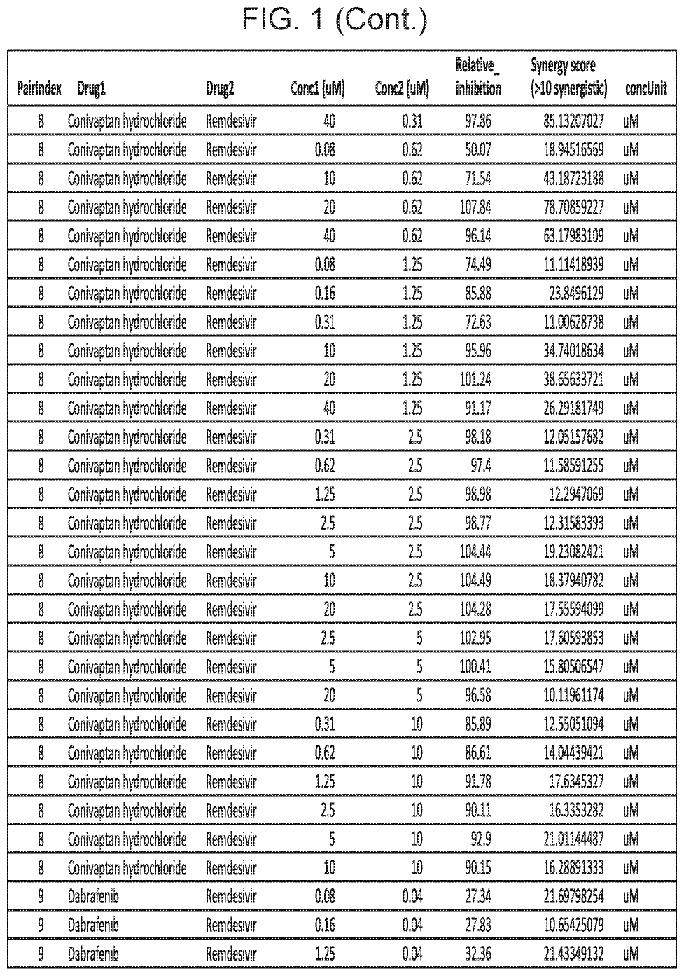

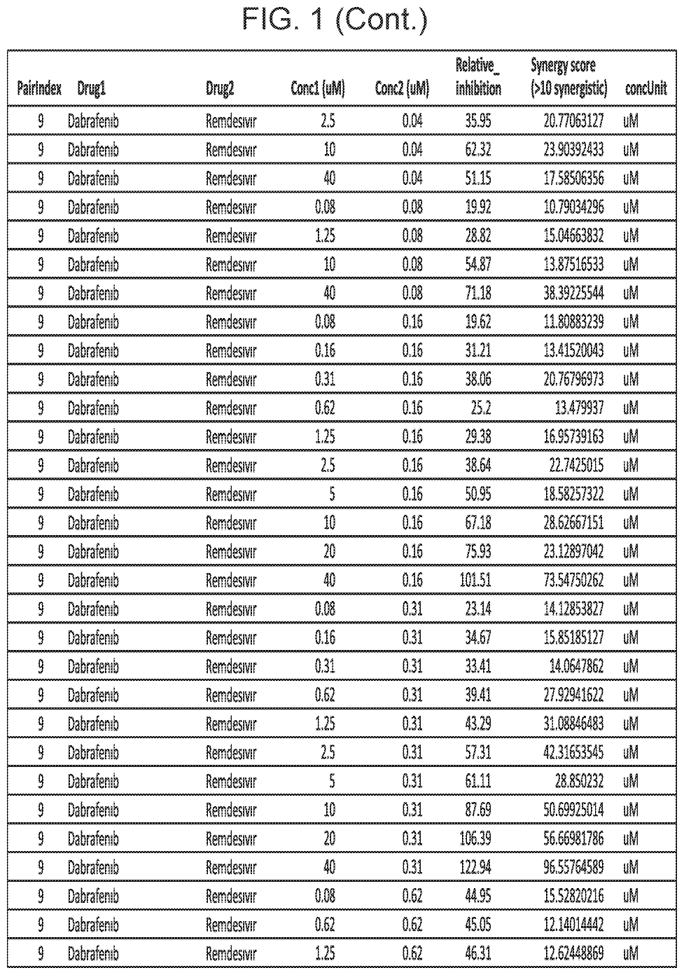

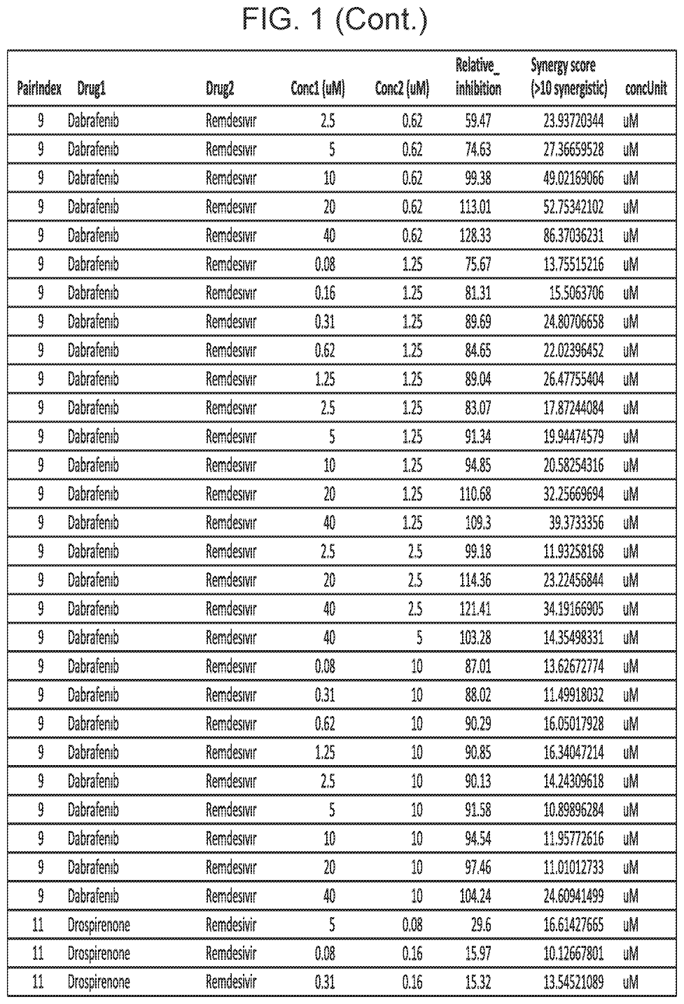

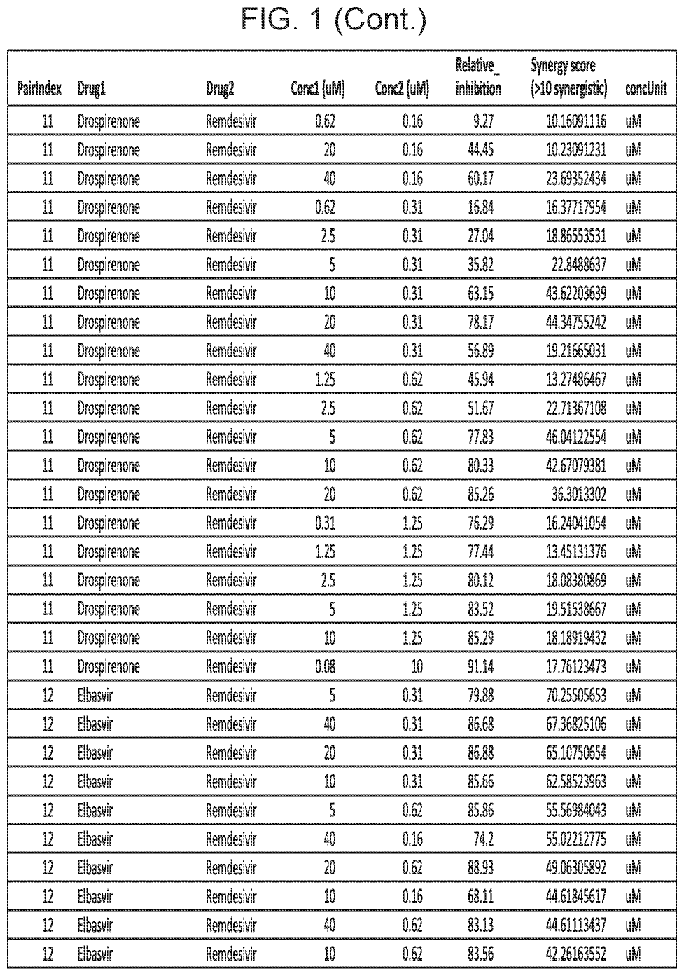

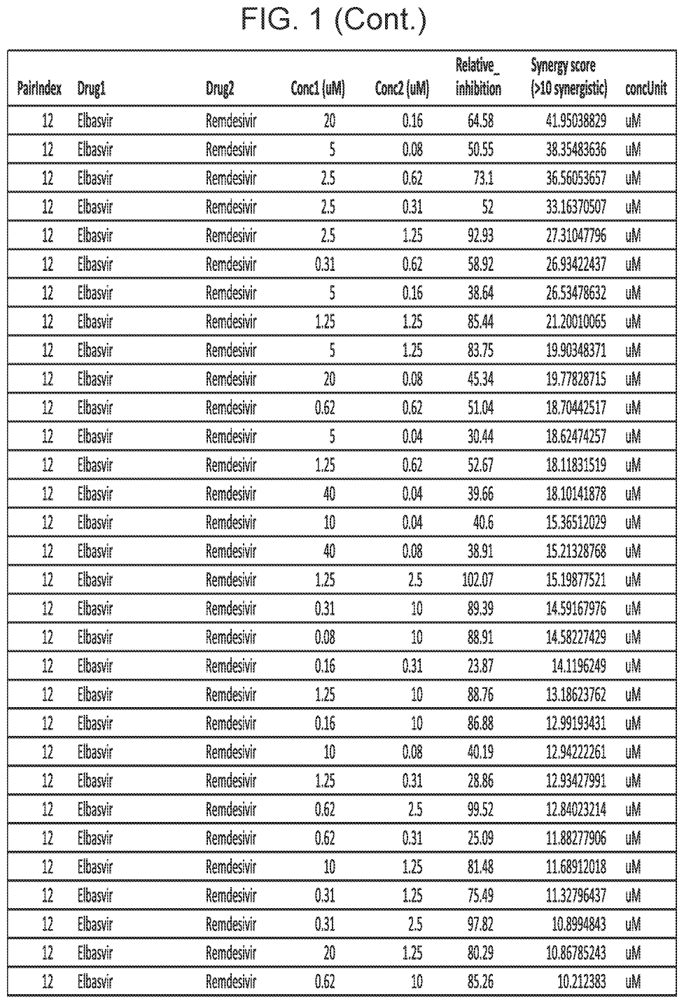

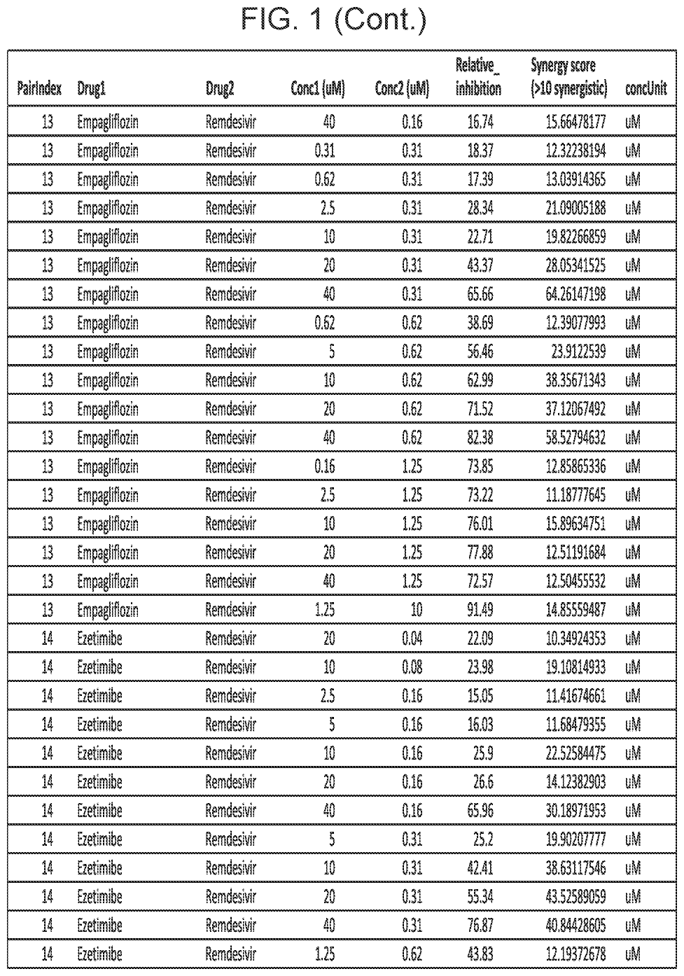

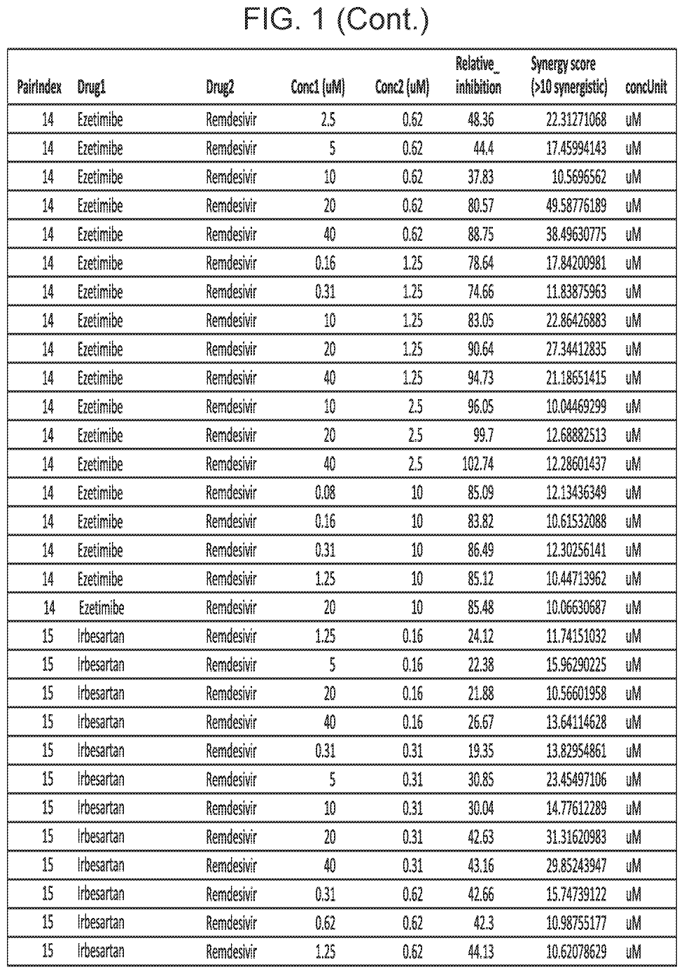

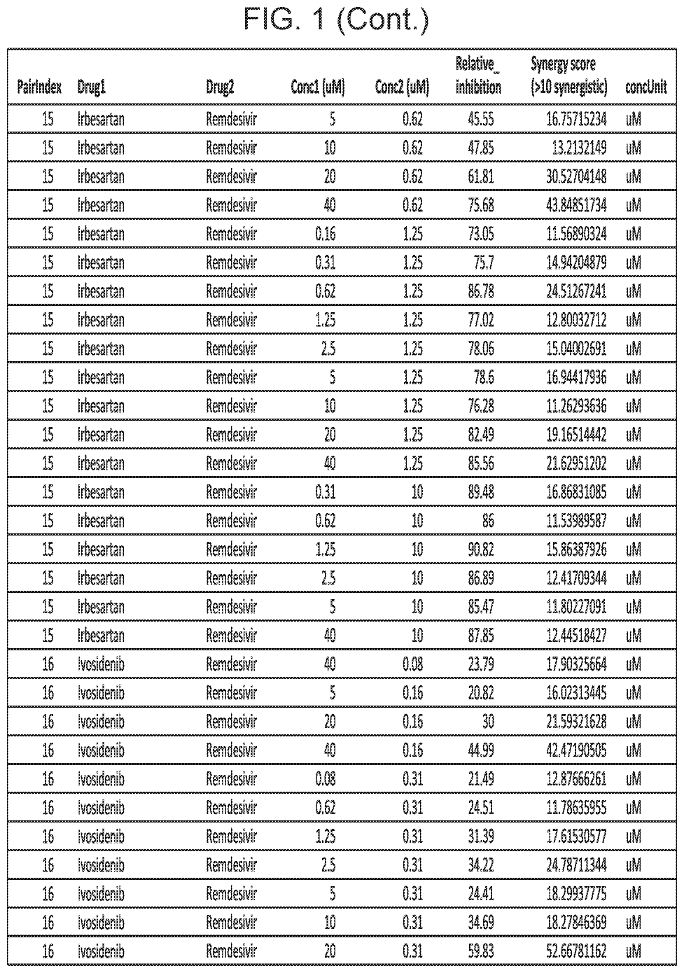

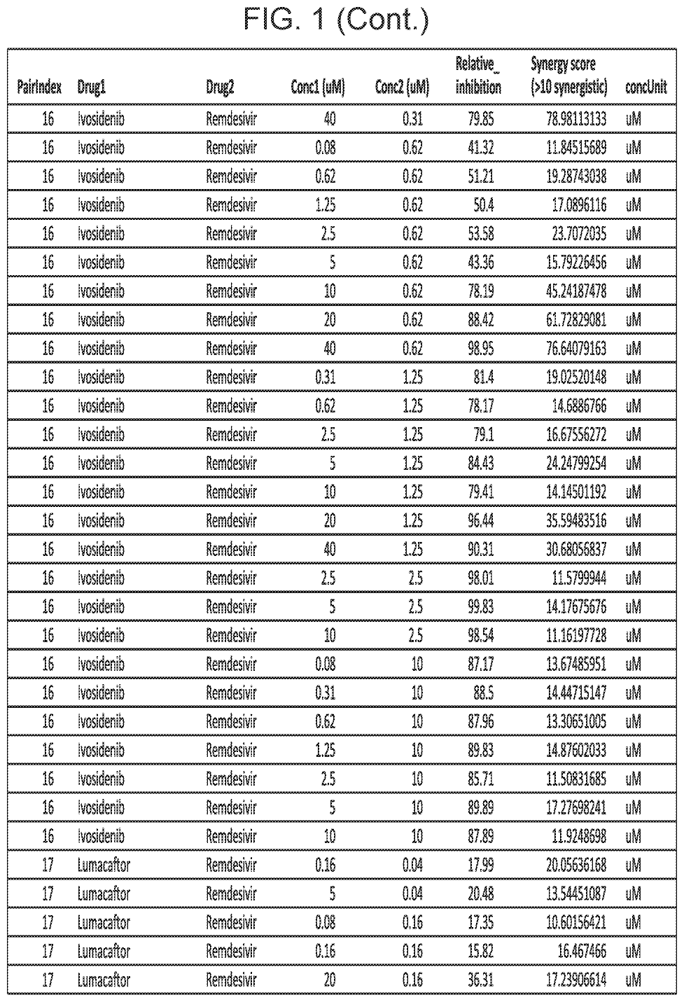

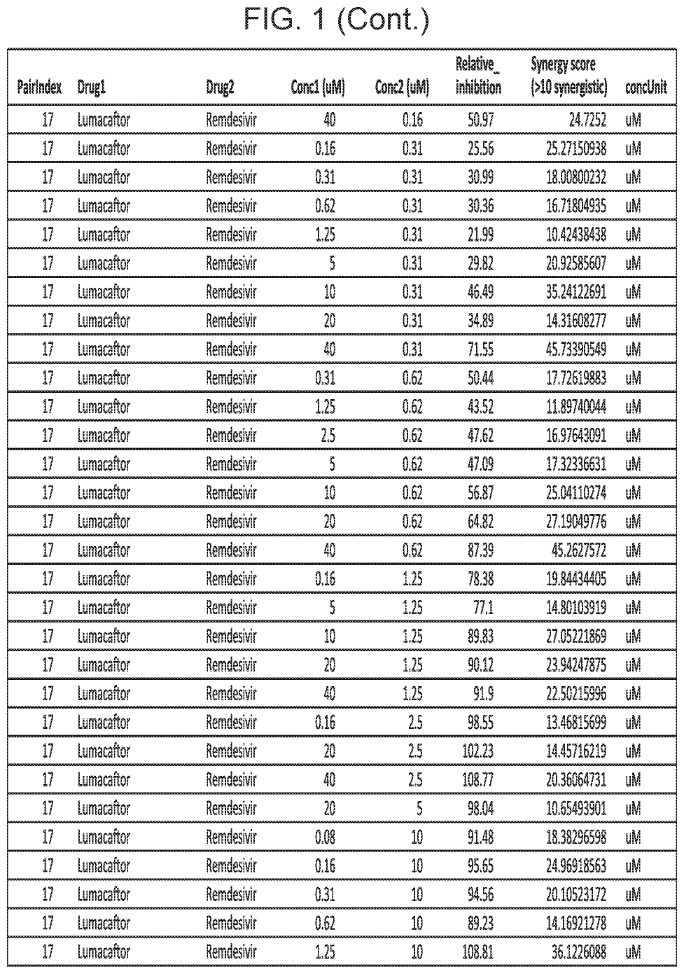

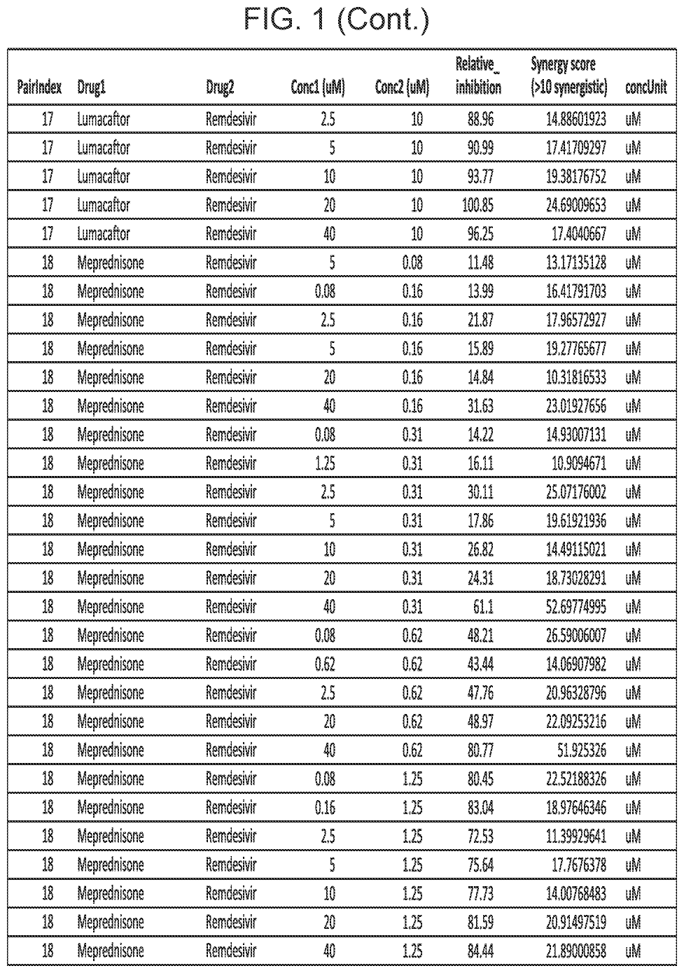

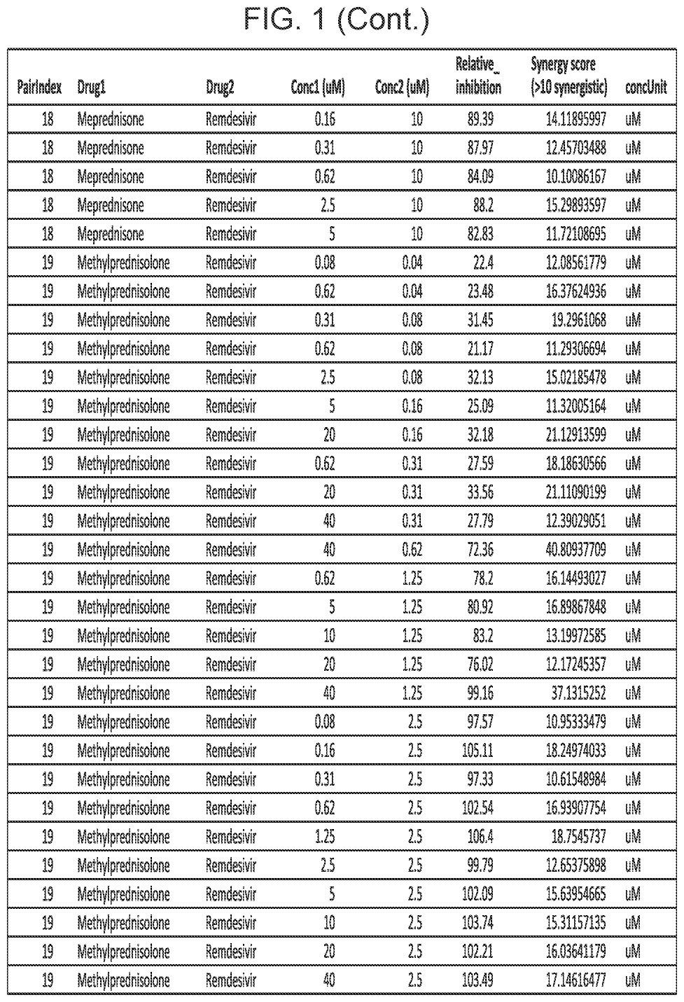

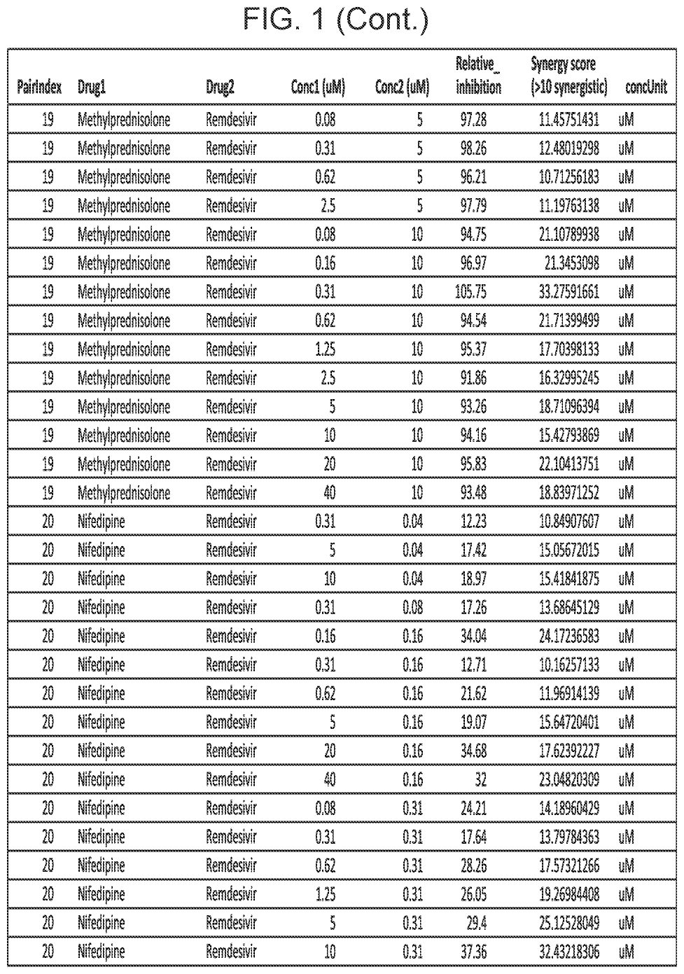

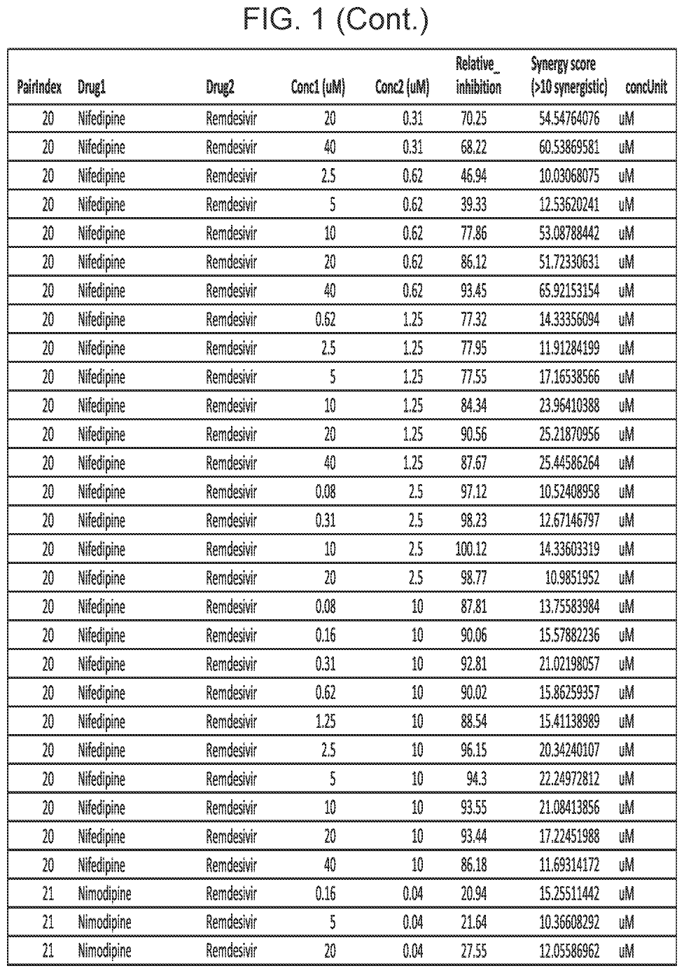

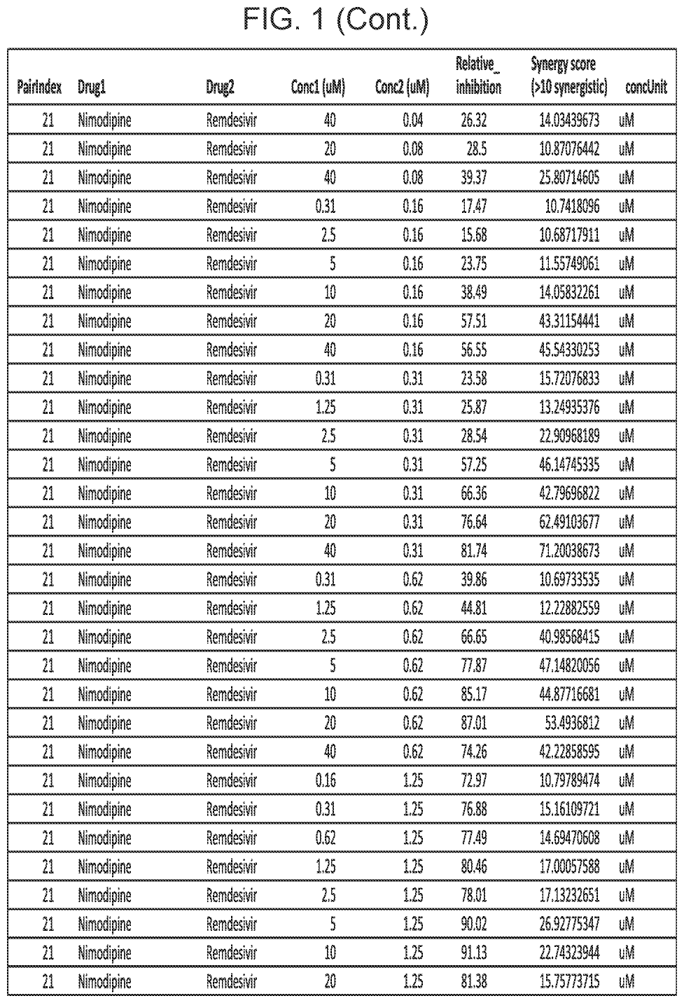

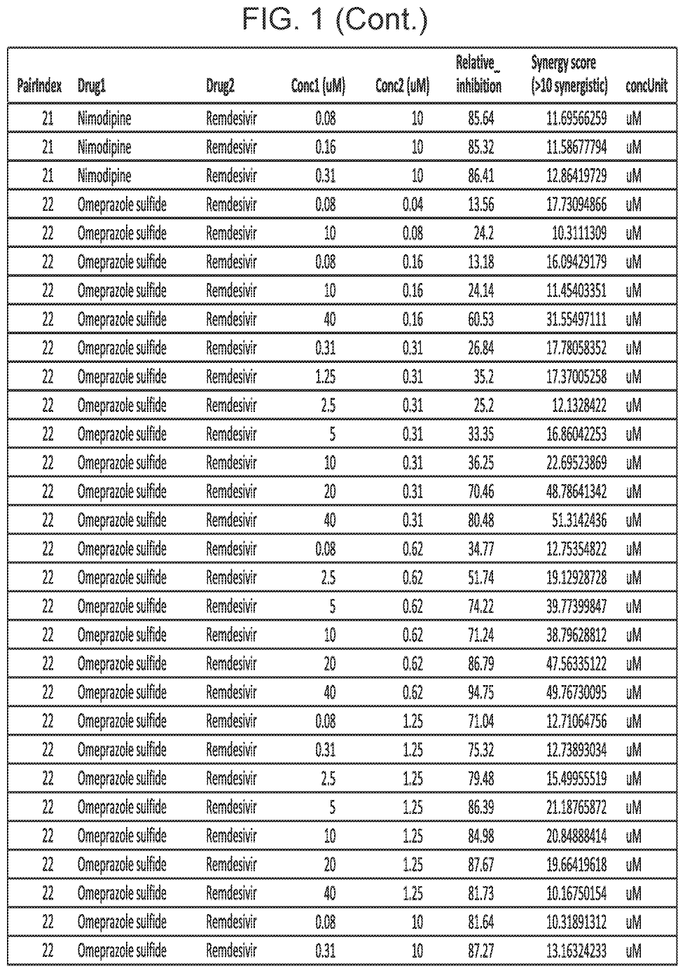

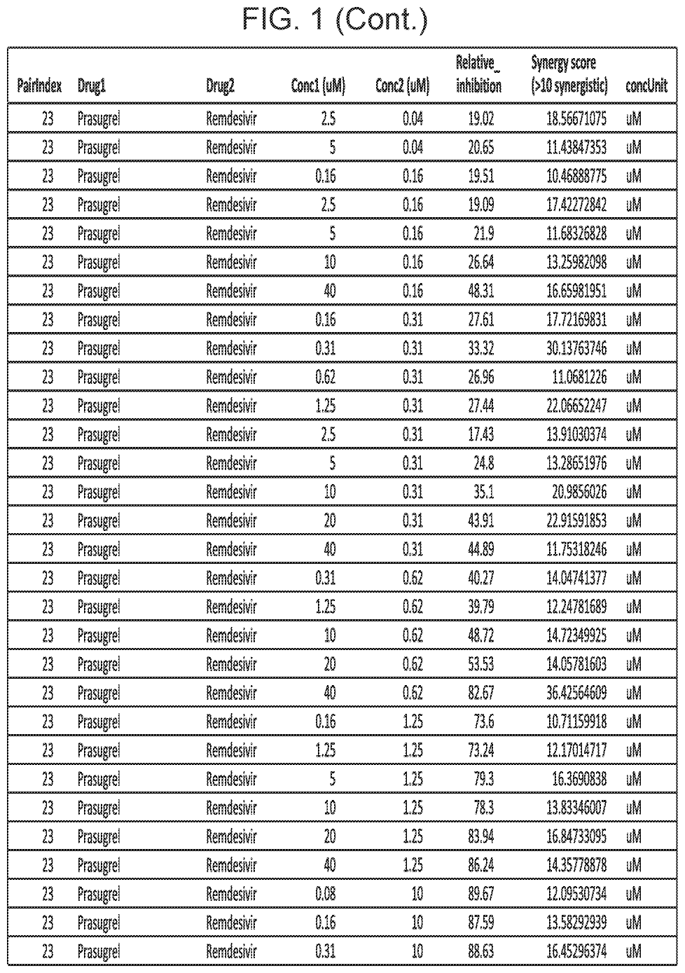

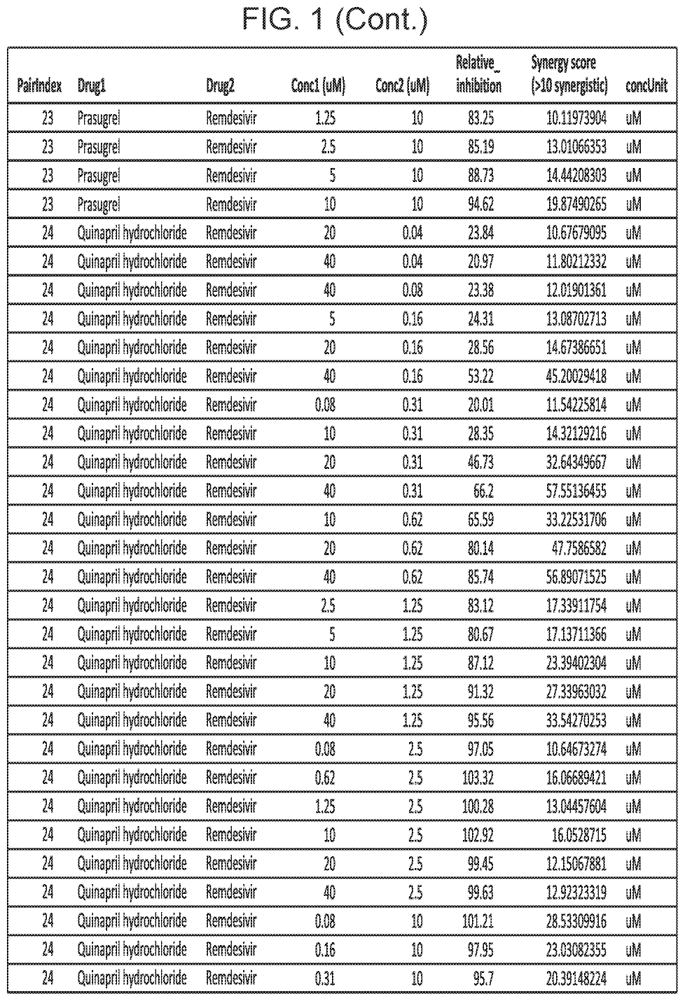

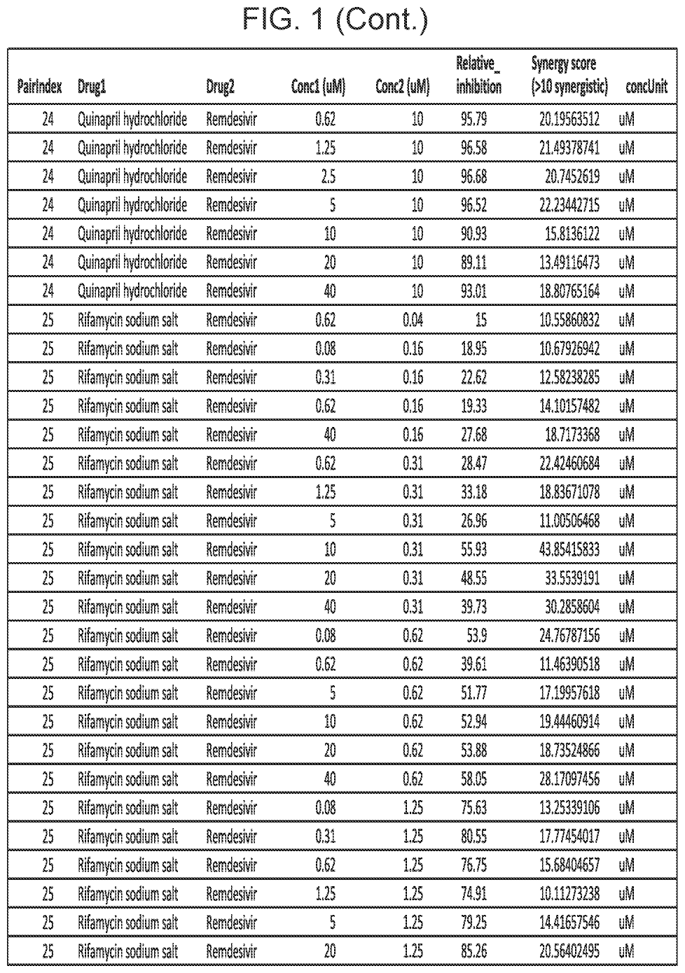

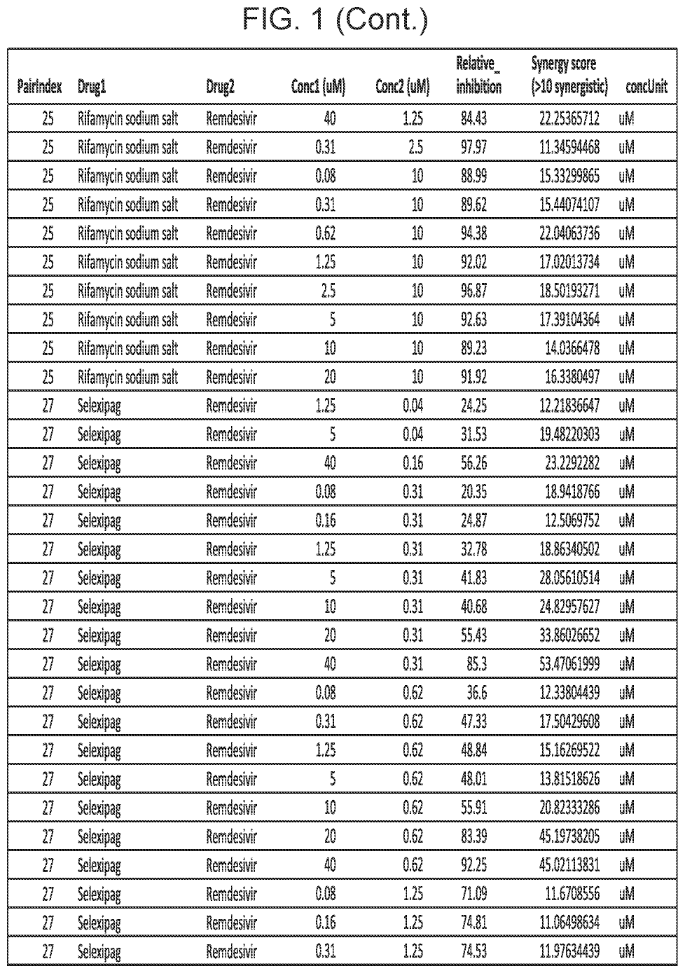

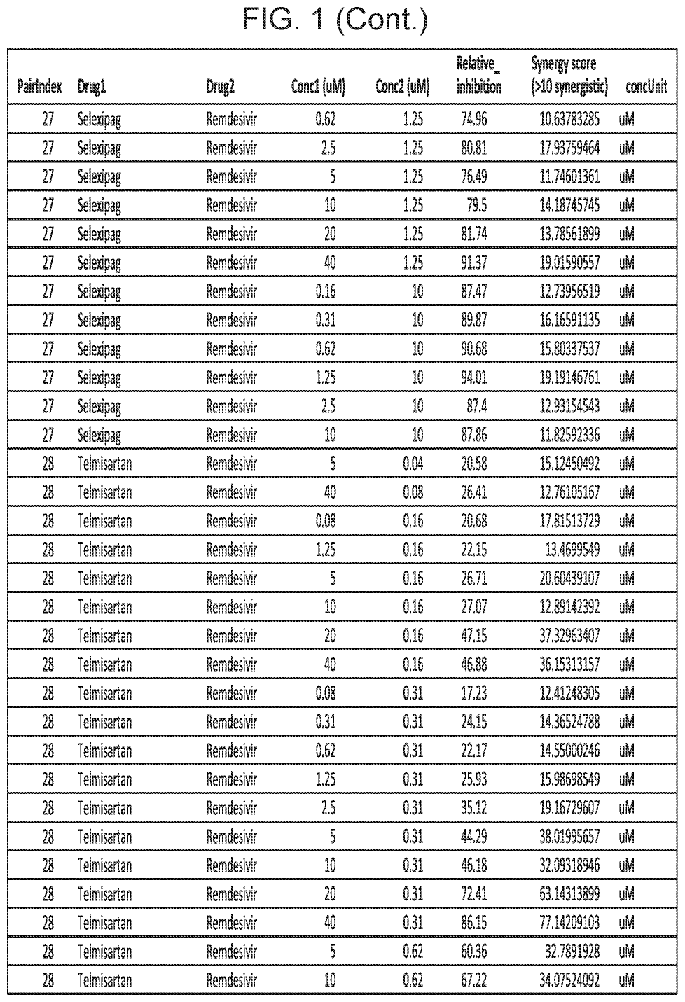

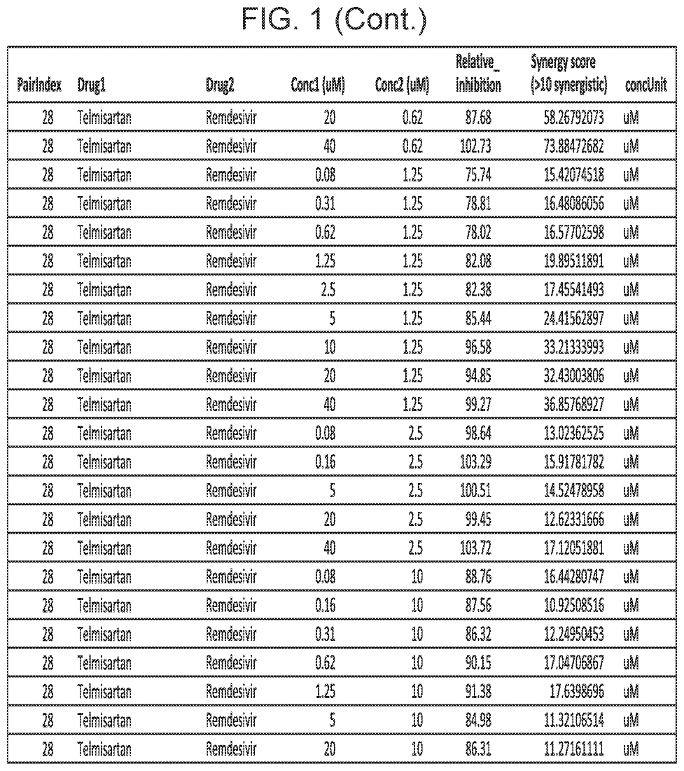

The results are shown in FIG. 1. FIG. 1 provides a full data list of synergy scores for all compounds mentioned (>10 signifies synergy, includes all combination concentration pairs that show synergy).

The synergy was strongest with Velpatasvir, an antiviral therapeutic currently used for Hepatitis C infection. Hepatitis C is an RNA virus. While Velpatasvir alone is of modest efficacy, and remdesivir is only active at high uM concentrations, strong synergistic effects were observed with the combination of Velpatasvir and remdesivir. For example, 300 nM remdesivir was barely active in the assay, preventing a virus-caused pathology in only 7% of cells. So is 5 M velpatasvir, inhibiting cytopathic effect in only 16% of cells. Administered together, efficacy increases to 100% in the cell-based model. Of note, also another Hepatitis C drug, Elbasvir, was synergistic with remdesivir, supporting the validity of the finding.

### Example 2

In combination with remdesivir, the following compounds or combinations of compounds were identified to exhibit synergy:
Sofosbuvir/velpatasvir/grazoprevir,
Sofosbuvir/velpatasvir/etravirine,
Sofosbuvir/velpatasvir/favipiravir,
Sofosbuvir/velpatasvir/ebselen,
Daclatasvir dihydrochloride,
Ledipasvir,
Mycophenolic acid,
ABT-530, Pibrentasvir,
Imatinib,
Ledipasvir acetone,
ABT-267, Ombitasvir.

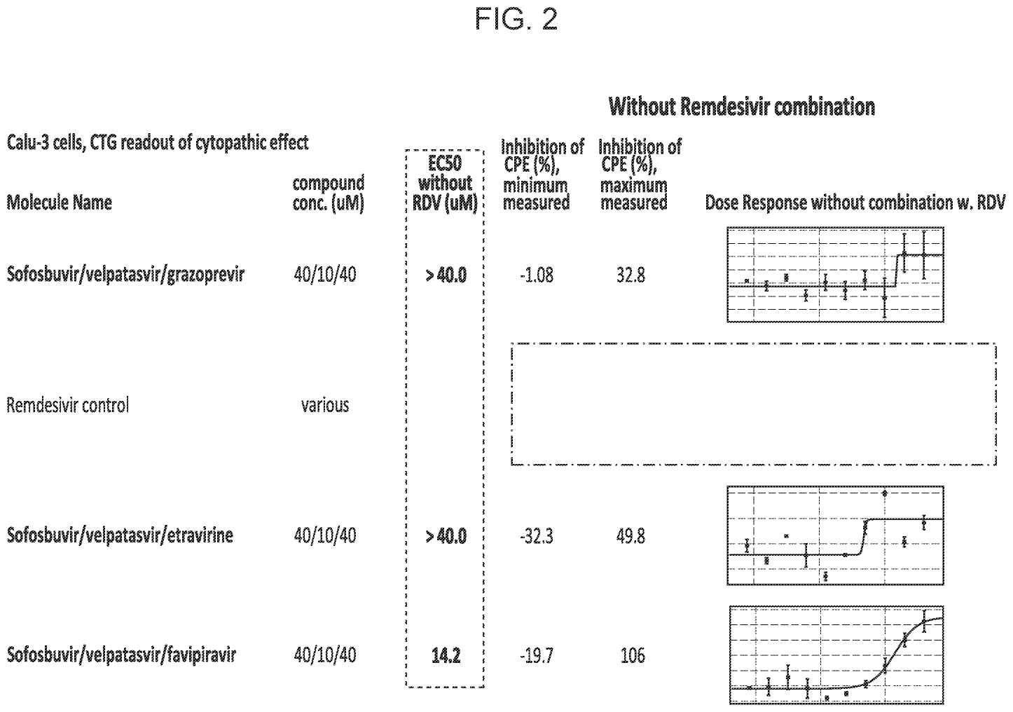

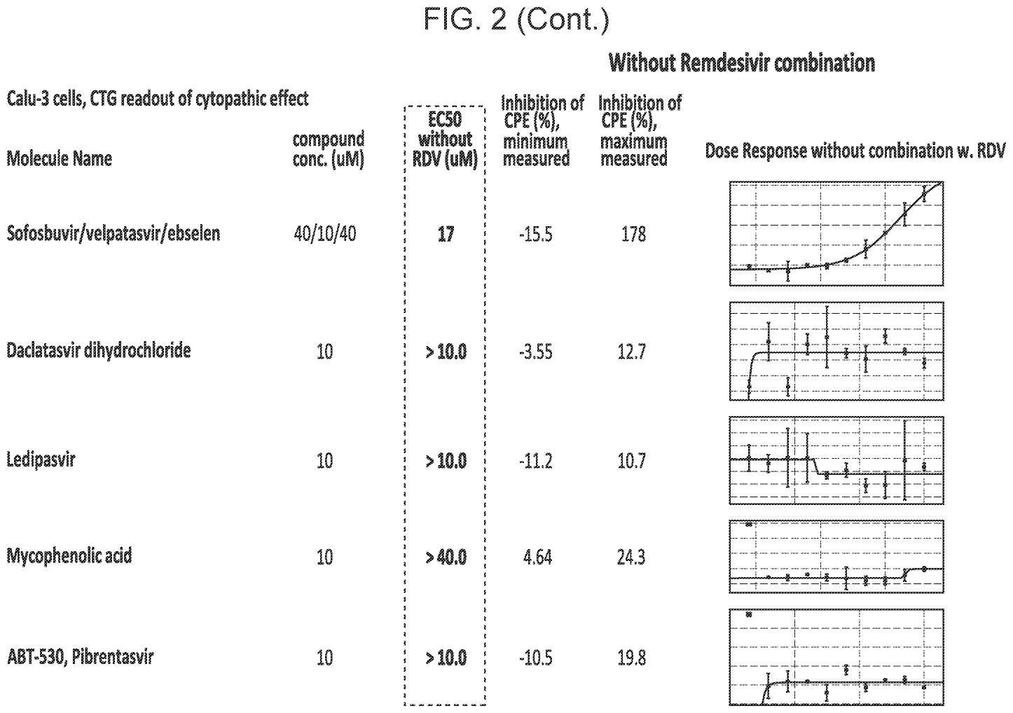

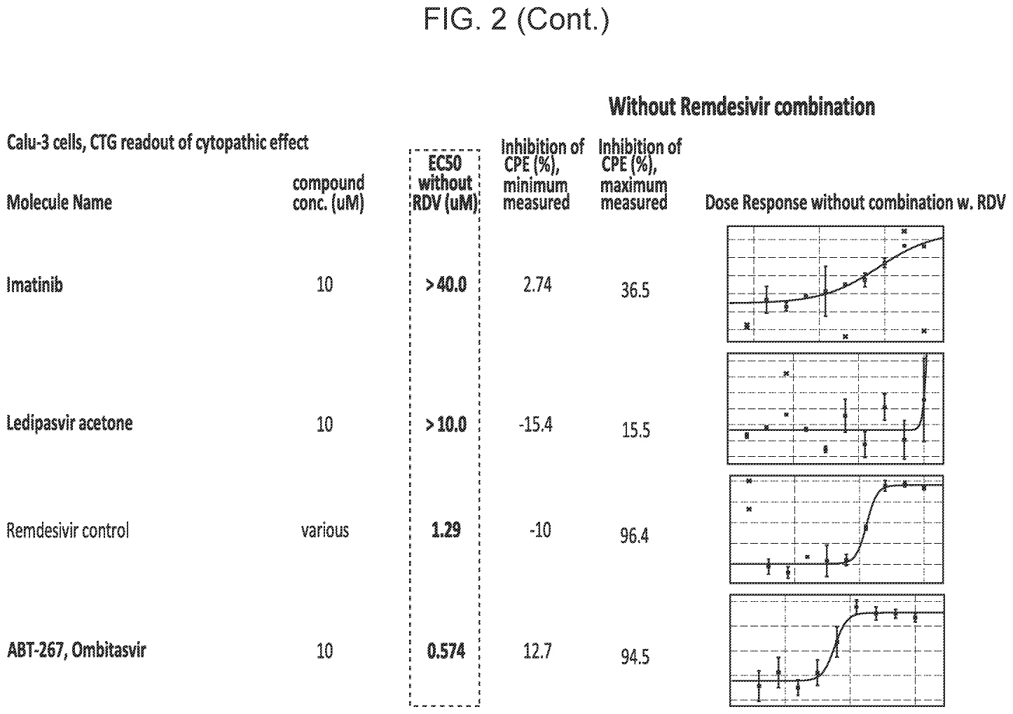

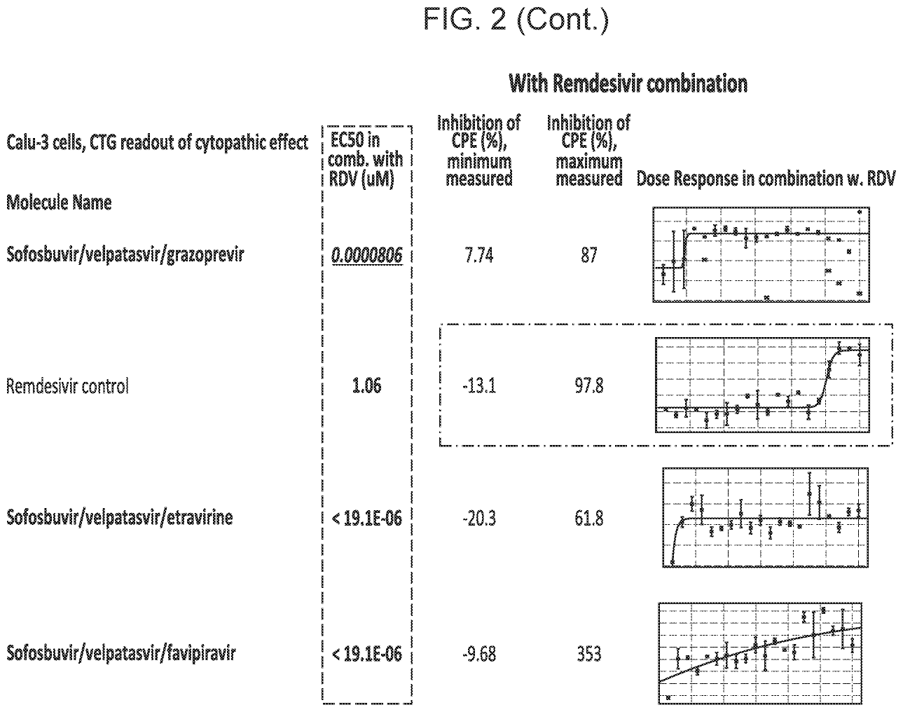

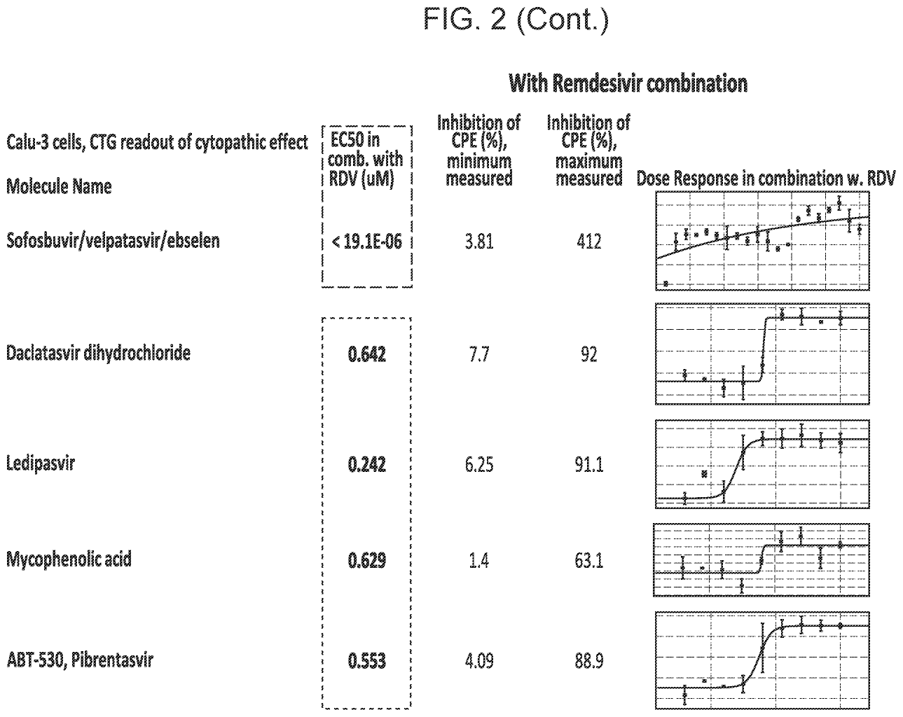

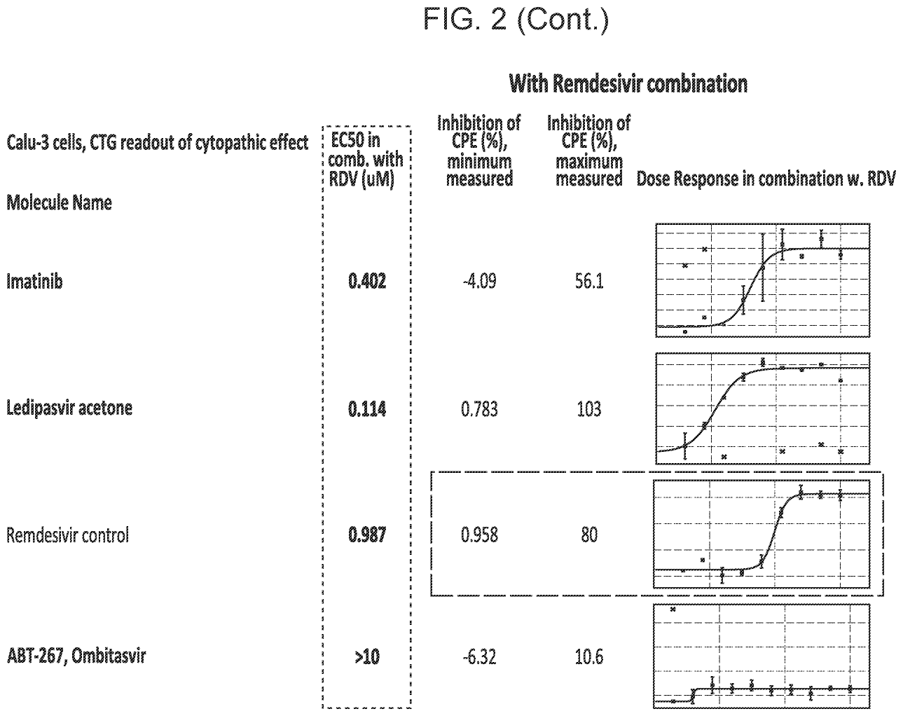

When Calu-3 cells were assayed for cytopathic effect caused by the virus, the compounds or combinations of compounds listed above exhibited higher percentage inhibition and lower EC50 when the cells were treated with these compounds or combinations of compounds along with remdisivir, compared to when the cells were treated with the compounds or combinations of compounds alone. (FIG. 2.) These data demonstrate that these compounds or combinations of compounds would be more effective at treating an infection with an RNA virus, such as SARS-CoV2 when they are administered in combination with remdesivir.

### Example 3

Combinations of agents were tested on four different strains of SARS-CoV-2.

### Materials and Methods

RNA extraction and real-time quantitative PCR (RT-qPCR). Calu-3 cells were plated and cultured for 2 days before the infection of SARS-CoV-2 (MOI 0.05). Infected Calu-3 cells (with medium) were lysed in DNA/RNA shield reagent (Zymo Research) and total RNA was extracted by using RNeasy kit (Qiagen) according to the manufacturer's protocol. cDNA was prepared by iScript™ Reverse Transcription Supermix (BioRAD) and qPCR was performed with Fast SYBR™ Green Master Mix (Thermo Fisher) and the reaction was run on the QuantStudio6 System (Applied Biosystems). mRNA levels were normalized to that of r18S.

qPCR primer sets were as follow:
SARS-COV-2 Nucleocapsid(N):
Fw
(SEQ ID NO: 1)
5′-GACCCCAAAATCAGCGAAAT-3′
and
Rv
(SEQ ID NO: 2)
5′-TCTGGTTACTGCCAGTTGAATCTG-3′,
SARS-COV-2 Spike(S):
Fw
(SEQ ID NO: 3)
5′-GTCCTTCCCTCAGTCAGCAC-3′
and
Rv
(SEQ ID NO: 4)
5′-ATGGCAGGAGCAGTTGTGAA-3′,
Human r18S:
Fw
(SEQ ID NO: 5)
5′-GTAACCCGTTGAACCCCATT-3′
and
Rv
(SEQ ID NO: 6)
5′-CCATCCAATCGGTAGTAGCG-3′,
Mouse r18S:
Fw
(SEQ ID NO: 7)
5′-GCAATTATTCCCCATGAACG-3′
and
Rv
(SEQ ID NO: 8)
5′-GGCCTCACTAAACCATCCAA-3′.

Analysis. For the ddCt value (calculation explained below) a value of 0.2 would mean an 80% reduction compared with dimethylsulfoxide (DMSO) control, it is the relative expression of XX to R18S. ddCt is called delta-delta C for RTqPCR. Ct is cycle number threshold of each sample/target in RTqPCR. It was calculated by software based on the amplification curve of PCR. RTqPCR amplification curve can be mimicked by f(x)=ax\hat{ }3+bx\hat{ }3+cx+d, where x is the Cycle number and y is the amount of PCR product. Ct is the cycle number that can give the maximal value of df(x)/dx=ax\hat{ }2+bx+c.

The delta-delta C assumes that with unlimited materials, an ideal primer set should have 100% amplification efficiency, which means 1 cycle of PCR will double the amount of PCR product. Given a Ct value of a sample, the original amount of a target in this sample is: Amount[target gene of Sample1]=2\hat{ }−Ct[target gene of Sample1]. First normalize this amount with expression level of r18S housekeeping gene to eliminate the different input amount among samples, so the normalization will be: Normalized Amount(target gene of Sample1)=2\hat{ }-Ct[target gene of Sample1]/2\hat{ }−Ct[r18S of Sample1]=2\hat{ }(Ct[r18S of Sample1]−Ct[target gene of Sample1]), also called 2\hat{ }−delta C[sample1]. Then one needs to compare the sample 1 with control, will be compared with control=2\hat{ }−delta C[sample1]/2\hat{ }−delta C[control]=2\hat{ }(delta C [control]−delta C[sample1]), also called 2\hat{ }-delta-delta C.

### Results

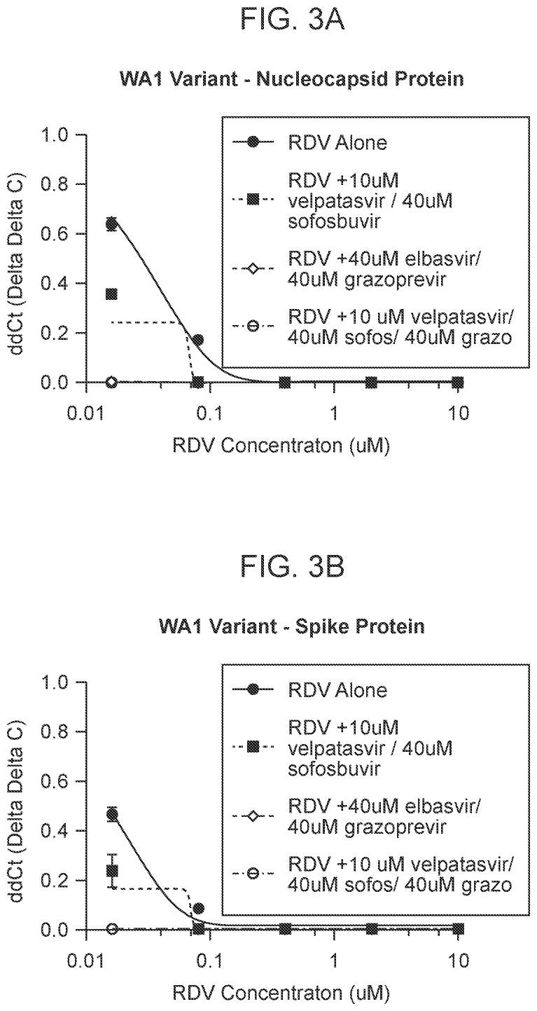

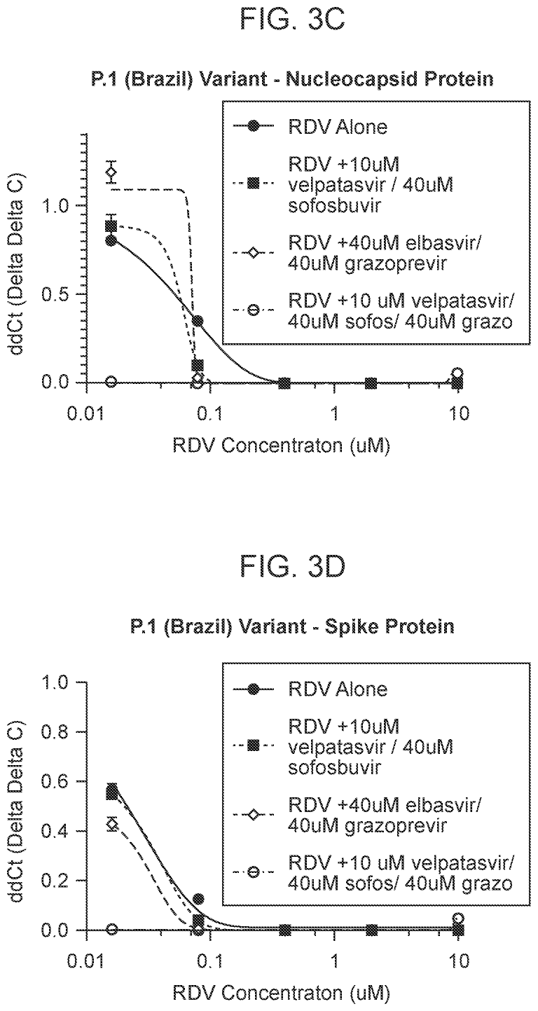

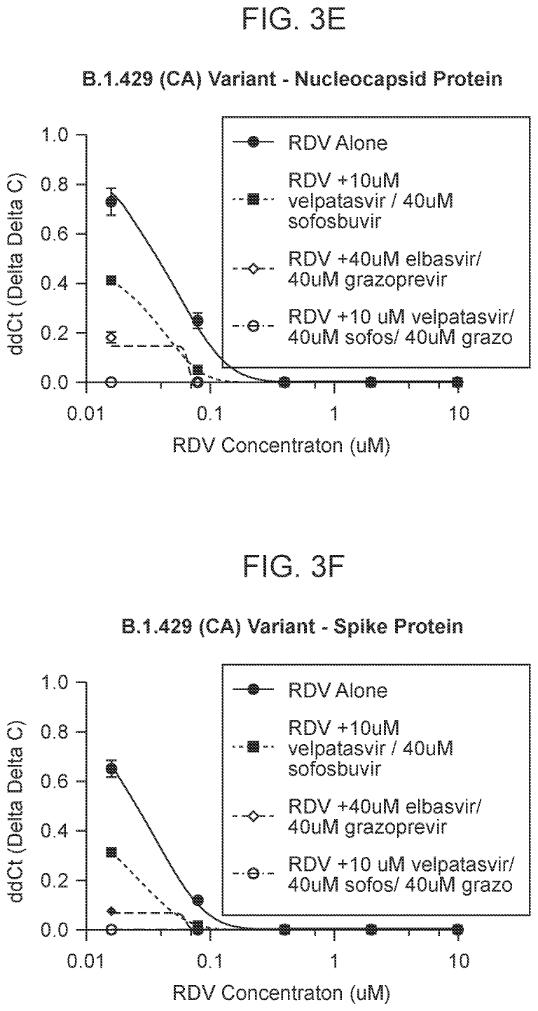

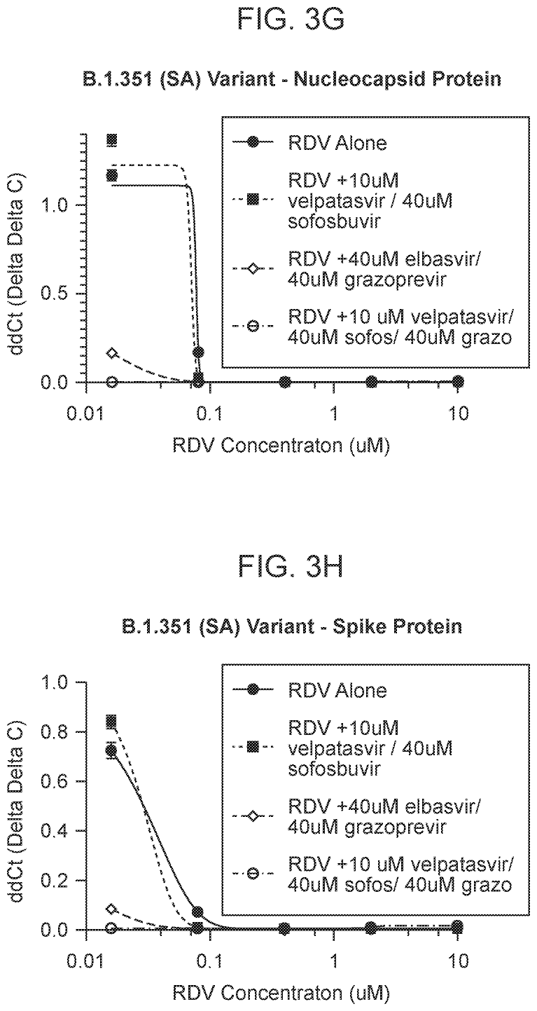

The results are shown in FIG. 3A-3H. The Nucleocapsid and the Spike genes were amplified in the RT-PCR readout. ddCt values were calculated with N=3. Ct is cycle number threshold of each sample/target in RTqPCR; it was calculated by software based on the amplification curve of PCR and it is the relative expression of sample to the R18S housekeeping gene. As shown in FIG. 3A-3H, combinations of agents, including the remdesivir/velpatasvir/grazoprevir combination, reduced the amount of the spike protein and the nucleocapsid protein.

FIG. 3A. (a) Dose response curve for Remdesivir (RDV) alone (solid circles) compared to RDV with a static dose of combination drugs at same concentration across all doses. The effect on WA1 variant, nucleocapsid protein, is shown.

FIG. 3B. Dose response curve for RDV alone (solid circles) compared to RDV with a static dose of combination drugs at same concentration across all doses. The effect on WA1 variant, spike protein, is shown.

FIG. 3C. Dose response curve for RDV alone (solid circles) compared to RDV with a static dose of combination drugs at same concentration across all doses. The effect on P.1 (Brazil) variant, nucleocapsid protein, is shown.

FIG. 3D. Dose response curve for RDV alone (solid circles) compared to RDV with a static dose of combination drugs at same concentration across all doses. The effect on P.1 (Brazil) variant, spike protein, is shown.

FIG. 3E. Dose response curve for RDV alone (solid circles) compared to RDV with a static dose of combination drugs at same concentration across all doses. The effect on B.1.429 (CA) variant, nucleocapsid protein, is shown.

FIG. 3F. Dose response curve for RDV alone (solid circles) compared to RDV with a static dose of combination drugs at same concentration across all doses. The effect on B.1.429 (CA) variant, spike protein, is shown.

FIG. 3G. Dose response curve for RDV alone (solid circles) compared to RDV with a static dose of combination drugs at same concentration across all doses. The effect on B.1.351 (SA) variant, nucleocapsid protein, is shown.

FIG. 3H. Dose response curve for RDV alone (solid circles) compared to RDV with a static dose of combination drugs at same concentration across all doses. The effect on B.1.351 (SA) variant, spike protein, is shown.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of treating a viral infection caused by an RNA virus, the method comprising administering a first therapeutic agent and at least one additional therapeutic agent in combined effective amounts, wherein:
a) the first therapeutic agent is an inhibitor of an RNA-dependent RNA polymerase (RdRp); and
b) the second therapeutic agent is an agent other than an RdRp inhibitor.

2. The method of claim 1, wherein the at least one additional therapeutic agent is selected from: a Hepatitis C Virus (HCV) inhibitor, an inhibitor of B-Raf, a proton pump inhibitor (PPI), an angiotensin II receptor blocker or antagonist, a prostacyclin receptor agonist, a calcium channel blocker, a dihydropyridine-type calcium channel blocker, a leukotriene receptor antagonist, a retinoid that selectively activates a retinoid X receptor, a corticosteroid, a selective inhibitor of phosphodiesterase type 3, an arginine vasopressin (AVP) receptor antagonist, an agonist of the progesterone receptor (PR), a selective inhibitor of cyclooxygenase-2, an inhibitor of the sodium glucose co-transporter-2 (SGLT-2), a Niemann-Pick C1-like 1 (NPC1L1) protein blocker, an inhibitor of isocitrate dehydrogenase-1 (IDH1), a protein folding chaperone, a corticosteroid, an antagonist of P2Y₁₂ adenosine diphosphate (ADP) receptor, and an angiotensin converting enzyme (ACE) inhibitor.

3. The method of claim 1 or claim 2, wherein the at least one additional therapeutic agent is selected from:
i) Sofosbuvir, velpatasvir, and grazoprevir;
ii) Sofosbuvir, velpatasvir, and etravirine;
iii) Sofosbuvir, velpatasvir, and favipiravir;
iv) Sofosbuvir, velpatasvir, and ebselen;
v) Daclatasvir dihydrochloride;
vi) Ledipasvir;
vii) mycophenolic acid;
viii) ABT-530 (Pibrentasvir);
ix) Imatinib;
x) Ledipasvir acetone; and
xi) ABT-267 (Ombitasvir).

4. The method of any one of claims 1-3, wherein the first therapeutic agent and the at least one additional therapeutic agent are administered in synergistically effective amounts.

5. The method of claim 2, wherein the HCV inhibitor is Velpatasvir.

6. The method of claim 1, wherein the HCV inhibitor is Elbasvir.

7. The method of claim 6, further comprising administering Grazoprevir, boceprevir, simeprevir, or elaprevir.

8. The method of claim 5 or claim 6, further comprising administering Sofosbuvir.

9. The method of claim 1, wherein the inhibitor of B-Raf is Dabrafenib.

10. The method of claim 1, wherein the PPI is Omeprazole.

11. The method of claim 1, wherein the angiotensin II receptor blocker is Telmisartan.

12. The method of claim 1, wherein the angiotensin II receptor antagonist is Irbesartan.

13. The method of claim 1, wherein the prostacyclin receptor agonist is Selexipag.

14. The method of claim 1, wherein the calcium channel blocker is Nifedipine or Nimodipine.

15. The method of claim 1, wherein the leukotriene receptor antagonist is Zafirlukast.

16. The method of claim 1, wherein the retinoid is Bexarotene.

17. The method of claim 1, wherein the PDE₃ inhibitor is Cilostazol.

18. The method of claim 1, wherein the AVP receptor antagonist is Conivaptan hydrochloride.

19. The method of claim 1, wherein the PR agonist is Drospirenone.

20. The method of claim 1, wherein the selective inhibitor of cyclooxygenase-2 is Valdecoxib.

21. The method of claim 1, wherein the SGLT-2 inhibitor is Empagliflozin.

22. The method of claim 1, wherein the NPC1L1 blocker is Ezetimibe.

23. The method of claim 1, wherein the IDH1 inhibitor is Ivosidenib.

24. The method of claim 1, wherein the protein folding chaperone is Lumacaftor.

25. The method of claim 1, wherein the corticosteroid is Meprednisone, Methylprednisolone, Budesonide, or Clobetasol propionate.

26. The method of claim 1, wherein the P2Y₁₂ ADP receptor antagonist is Prasugrel.

27. The method of claim 1, wherein the ACE inhibitor is Quinapril hydrochloride.

28. The method of any one of claims 1-27, wherein the RdRp inhibitor is sofosbuvir, remdesivir, ribavirin, favipiravir, pimodivir, or baloxavir.

29. The method of any one of claims 1-27, wherein the RdRp inhibitor is remdesivir.

30. The method of any one of claims 1-28, wherein the virus is SARS-CoV2.

31. The method of claim 30, further comprising administering an antibody specific for the SARS-CoV2 spike glycoprotein.

32. The method of claim 31, further comprising administering famotidine.

33. The method of any one of claims 30-32, wherein the individual has been diagnosed as having Covid-19.

34. The method of any one of claims 30-32, wherein the individual exhibits one or more symptoms of a SARS-CoV2 infection.

35. The method of any one of claims 30-34, wherein the individual has an oxygen saturation of less than 94%.

36. The method of any one of claims 30-35, wherein the individual is receiving supplemental oxygen.

37. The method of any one of claims 30-35, wherein the individual requires mechanical ventilation or extracorporeal membrane oxygenation.

38. The method of any one of claims 30-37, wherein the individual weighs 40 kg or more, and remdesivir is administered in a single loading dose of 200 mg on Day 1 followed by a once-daily maintenance dose of 100 mg from Day 2.

39. The method of any one of claims 30-37, wherein the individual weighs 3.5 kg to 40 kg, and remdesivir is administered in a single loading dose of 5 mg/kg on Day 1, followed by a once daily dose of 2.5 mg/kg from Day 2.

40. The method of claim 38 or claim 39, wherein the dose of remdesivir is reduced by from 10% to 50%.

41. The method of any one of claims 38-40, wherein the remdesivir is administered intravenously.

42. The method of any one of claims 30-41, wherein the SARS-CoV2 is a variant SARS-CoV2 selected from the B.1.1.7 variant, the B.1.351 variant, the B.1.617.2 variant, and the P.1 variant.

43. The method of any one of claims 30-42, wherein the RdRP inhibitor is remdesivir, and wherein the method comprises administering combined effective amounts of remdesivir, velpatasvir, and grazoprevir.
